SEQUENCE LISTING (10 sequences)

SEQ ID NO: 1 - protein, 36 aa, Leiurus quinquestriatus
  MISC_FEATURE (1)..(36): Chlorotoxin peptide
MCMPCFTTDHQMARKCDDCCGGKGRGKCYGPQCLCR

SEQ ID NO: 2 - protein, 35 aa, Buthus sindicus
  MISC_FEATURE (1)..(35): Bs-Tx7 peptide
CGPCFTTDWESEKKCAECCGGIGRCFGPQCLCNRK

SEQ ID NO: 3 - protein, 35 aa, Buthus sindicus
  MISC_FEATURE (1)..(35): Bs-Tx8 peptide
RCKPCFTTDPQMSKKCADCCGGKGKGKCYGPQCLC

SEQ ID NO: 4 - protein, 36 aa, Buthus sindicus
  MISC_FEATURE (1)..(36): Bs-Tx14
CGPCFTKDPETEKKCATCCGGIGRCFGPQCLCNRGY

SEQ ID NO: 5 - protein, 34 aa, Leiurus quinquestriatus
  MISC_FEATURE (1)..(34): GaTx1 peptide
CGPCFTTDHQMEQKCAECCGGIGKCYGPQCLCNR

SEQ ID NO: 6 - protein, 34 aa, Leiurus quinquestriatus
  MISC_FEATURE (1)..(34): CLTx-a peptide
CMPCFTTDHQMARKCDDCCGGRGKCYGPQCLCRG

SEQ ID NO: 7 - protein, 34 aa, Leiurus quinquestriatus
  MISC_FEATURE (1)..(34): CLTx-b peptide
CGPCFTTDHQTEQKCAECCGGIGKCYGPQCLCRG

SEQ ID NO: 8 - protein, 34 aa, Leiurus quinquestriatus
  MISC_FEATURE (1)..(34): CLTx-c peptide
CGPCFTTDRQMEQKCAECCGGIGKCYGPQCLCRG

SEQ ID NO: 9 - protein, 32 aa, Leiurus quinquestriatus
  MISC_FEATURE (1)..(32): CLTx-D peptide
CGPCFTTDHQTEQKCAECCGGIGKCYGPQCLC

SEQ ID NO: 10 - protein, 35 aa, Buthus martensii
  MISC_FEATURE (1)..(35): BmKCL1 peptide
CGPCFTTDANMARKCRECCGGIGKCFGPQCLCNRI

(19) United States
(12) Patent Application Publication
(10) Pub. No.: US 2019/0076555 A1
(43) Pub. Date: Mar. 14, 2019

(54) TUMOR TARGETING NANOAGENT FOR IMAGING AND FLUORESCENT GUIDED RESECTION OF TUMORS

(71) Applicant: Cedars-Sinai Medical Center, Los Angeles, CA (US)

(72) Inventors: Rameshwar Patil, Los Angeles, CA (US); Eggehard Holler, Los Angeles, CA (US); Julia Y. Ljubimova, Studio City, CA (US); Adam Mamelak, Sherman Oaks, CA (US); Keith L. Black, Los Angeles, CA (US)

(73) Assignee: Cedars-Sinai Medical Center, Los Angeles, CA (US)

(21) Appl. No.: 16/128,888

(22) Filed: Sep. 12, 2018

Related U.S. Application Data
(60) Provisional application No. 62/557,380, filed on Sep. 12, 2017.

(51) Int. Cl. A61K 49/00; A61K 49/18

(52) U.S. Cl. CPC A61K 49/0032; A61K 49/0002; B82Y 30/00; A61K 49/186; A61K 49/0093

(57) ABSTRACT

Imaging nanoagents including a polymalic acid-based molecular scaffold, a chlorotoxin peptide or a variant thereof, and at least one fluorescent moiety are provided. Methods for detecting, treating and removing a cancer in a subject by administering the imaging nanoagent are described.

### CROSS REFERENCE TO RELATED APPLICATION

This application claims the benefit of U.S. provisional application No. 62/557,380, filed Sep. 12, 2017, which is incorporated herein by reference as if fully set forth.

### STATEMENT REGARDING FEDERALLY SPONSORED RESEARCH OR DEVELOPMENT

The invention was made with government support under Grant No. CA209921-01 awarded by National Institutes of Health. The government has certain rights in the invention.

The sequence listing electronically filed with this application titled “Sequence Listing,” which was created on Sep. 12, 2018 and had a size of 4,722 bytes is incorporated by reference herein as if fully set forth.

### FIELD OF INVENTION

The disclosure generally relates to imaging nanoagents that include polymalic acid-based scaffold, chlorotoxin peptide or a variant thereof and at least one fluorescent moiety attached to the polymalic-acid scaffold. The disclosure also relates to methods for fluorescent guided resection of tumors in patients having cell proliferative disorders by administering the imaging nanoagents and compositions comprising the same to the patients.

### BACKGROUND

Despite significant efforts and a wealth of new data on glioma biology, the patients' survival did not significantly change in the last 25 years (Noone et al. (eds). SEER Cancer Statistics Review, 1975-2015, National Cancer Institute. Bethesda, Md.; Deorah et al., 1973 to 2001. Neurosurg. Focus, 2006; 20:E1; Chi et al. Neurotherapeutics. 2009; 6:513-526). The National Cancer Institute estimated that 23,880 malignant brain and spinal cord tumors were diagnosed in 2018 in the U.S. Gliomas are the most common brain malignancies, and a very aggressive tumor, glioblastoma grade IV (glioblastoma multiforme, or GBM), is the most frequently occurring glioma. Resection has remained the major treatment. However, its success depends on the extent of the resection obtained. Even in the best cases, gliomas are not completely separable from the normal brain due to deep infiltration of malignant cells within the normal brain parenchyma. Therefore, there is an unmet clinical need in a combination of treatments involving: first, the best possible resection; and second, the elimination of residual glioma cells based on specific markers to suppress tumor regrowth.

There are several formidable obstacles to the development of effective and long-lasting therapies. These obstacles include: 1) the infiltrative nature of gliomas, typically growing many centimeters into surrounding viable brain; 2) the difficulty in visually differentiating normal brain parenchyma from the infiltrating tumor; 3) the functional organization of the brain that prevents removal of large areas without major neurological consequences; 4) the relative chemotherapeutic resistance of brain tumors; and 5) the low therapeutic to toxic ratio of radiation therapy in the brain. Each one of these issues is somewhat unique to brain tumors, and therefore, resolving each of them is critical to the development of effective treatments. Nanomedicines targeting tumor-specific ligands that can deliver therapies with great precision represent a highly promising approach to overcoming these limitations.

Despite decades of efforts to develop effective chemotherapies and radiation therapies, surgery remains the single most successful strategy for the treatment of gliomas (Hervey-Jumper and Berger, Curr Treat Options Neurol. 2014; 16:284; Ius et al., J Neurosurg. 2012; 117:1039-1052, which are incorporated herein by reference as if fully set forth). The utility of surgery is highly dependent on the extent of resection obtained, with increased survival demonstrated when >95% of the enhancing tumor volume is resected (Eyipoglu et al., Nat Rev Neurol. 2013; 9:141-151; Bloch et al. J Neurosurg. 2012; 117:1032-1038, which are incorporated herein by reference as if fully set forth). Recent data have demonstrated a significant survival rate without increased morbidity when the surrounding FLAIR signal from MRI is also resected (Beiko et a. Neuro Oncol. 2014; 16:81-91, which is incorporated herein by reference as if fully set forth).

Several strategies have been employed to improve the extent of resection while limiting damage to surrounding brain tissue including intraoperative MRI, and induced tumor fluorescence (Kubben et al., Lancet Oncol. 2011; 12:1062-1070, which is incorporated herein by reference as if fully set forth). To date, the most successful of these methods is the use of 5 Amino Levulenic Acid (5-ALA) to induce fluorescence in tumor cells by driving mitochondrial protoporphyrin IX (pP IX) synthesis, followed by subsequent detection of pP IX in the ultraviolet (UV, 485 nm) light range (Kubben et al., Lancet Oncol. 2011; 12:1062-1070; Hefti et al., Swiss Med Wkly. 2008; 138:180-185; Pichlmeier et al., Neuro-Oncology. 2008; 10:1025-1034; Stummer et al., Lancet Oncology. 2006; 7:392-401, all of which are incorporated herein by reference as if fully set forth.). Despite many drawbacks of pP IX as a fluorescent marker, including the non-specificity, “bleeding” of fluorescence into normal tissue due to cell lysis, poor tissue penetration, poor signal to noise ratio, and side effects from 5-ALA administration, this method has been demonstrated to improve the extent of resection and subsequent progression-free survival (Chung and Eljamel, Photodiagnosis Photodyn Ther. 2013; 10:362-367; Eyiipoglu et al. PLoS One. 2012; 7:e44885; Stummer et al., Lancet Oncol. 2006; 7:392-401, which are incorporated herein by reference as if fully set forth).

Recent interest has focused on the use of near infrared (NIR) rather than UV fluorescence. NIR has greater spatial resolution than UV light, and a narrow emission spectrum permitting filter optimization for fluorescence detection. There is little absorption by hemoglobin and minimal light scattering in these wavelengths, so intervening normal tissue does not attenuate the signal to the same extent seen with other wavelengths (Thurber et al., Journal of Surgical Oncology. 2010; 102:758-764, which is incorporated herein by reference as if fully set forth).

Finally, there is very low tissue auto-fluorescence at the NIR emission wavelength, enabling very good signal to noise and sharp definition of tumor boundaries.

### SUMMARY

In an aspect, the invention relates to an imaging nanoagent. The imaging nanoagent comprises a polymalic acid-based molecular scaffold, a chlorotoxin peptide or variant thereof, and at least one fluorescent moiety. The chlorotoxin peptide and the at least one fluorescent moiety are covalently linked to the polymalic acid-based molecular scaffold.

In an aspect, the invention relates to a pharmaceutically acceptable composition comprising any one of the imaging nanoagents described herein and a pharmaceutically acceptable carrier or excipient.

In an aspect, the invention relates to a method for detecting and removing a cancer. The method comprises administering any one of the imaging nanoagents described herein or a pharmaceutically acceptable composition described herein to a subject to detect cancerous cells or tissue. The method comprises detecting the presence or absence of the imaging nanoagent, wherein the presence of the imaging nanoagent in the cells or tissues indicates the presence of cancerous cells or tissue. The method also comprises surgically removing the cancerous cell or tissue.

In an aspect, the invention relates to a method of imaging a tissue in a brain of a subject. The method comprises administering any one of the imaging nanoagents described herein or any one of the pharmaceutically acceptable compositions described herein to a subject in need thereof. The method further comprises visualizing the imaging nanoagent.

In an aspect, the invention relates to a method for treating cancer in a subject. The method comprises administering any one of the imaging nanoagents described herein or any one of the pharmaceutically acceptable compositions described herein to a subject in need thereof.

In an aspect, the invention relates to an imaging nanoagent. The imaging nanoagent comprises a polymalic acid-based molecular scaffold, a chlorotoxin peptide covalently linked to the polymalic acid-based molecular scaffold by a polyethylene glycol (PEG) linker, a plurality of cyanine moieties covalently linked to the polymalic acid-based molecular scaffold, at least one biologically active molecular module covalently linked to the polymalic acid-based molecular scaffold. The at least one biologically active molecular module is selected from the group consisting of an anti-cancer agent, a targeting ligand, and an endosomolytic ligand, and the plurality of the cyanine moieties are interspaced with the chlorotoxin peptide, the at least one biologically active molecular module, or a combination thereof.

### BRIEF DESCRIPTION OF THE DRAWINGS

The patent or application file contains at least one color drawing or photograph as a drawing executed in color. Copies of this patent or patent application publication with color drawing(s) will be provided by the Office upon request and payment of the necessary fee.

The following detailed description of the preferred embodiments will be better understood when read in conjunction with the appended drawings. For the purpose of illustration, there are shown in the drawings embodiments which are presently preferred. It is understood, however, that the invention is not limited to the precise arrangements and instrumentalities shown. In the drawings:

FIGS. 1A-1B are schematic drawings of the imaging nanoagents that include polymalic acid (P) conjugated to Iodocyanine Green (IGC) (FIG. 1A), and P conjugated to IGC and Chlorotoxin (CTX) (FIG. 1B). FIG. 1A illustrates the schematic drawings of control imaging nanoagents consisting of a polymalic acid (P) with 10 pendent carboxylic groups covalently conjugated with ICG: the structure on the left represents polymalic acid (P) conjugated to IGG (2%), and the structure on the right represents polymalic acid (P) conjugated to ICG (2%) and tri-leucine (LLL) (40%). FIG. 1B illustrates the schematic drawings of tumor specific imaging nanoagents similar to the control molecules shown on FIG. 1A but additionally possessing the tumor specific targeting ligand CTX (1.5%) that is covalently attached via PEG linker to the polymalic acid (P) to ensure high integrity of the imaging nanoagents.

FIGS. 2A-2D illustrate synthesis of imaging nanoagents and intermediates. FIG. 2A illustrates attachment of PEG linker to CTX and formation of CTX-PEG2000-MAL. FIG. 2B illustrates commercially available ICG-Maleimide (ICG-MAL). FIGS. 2C-2D illustrate synthesis of polymalic acid-based imaging nanoagents P/ICG(2%), P/CTX(1.5%)/ICG (2%) (FIG. 2C) and P/LLL(40%)/ICG(2%), P/LLL(40%)/CTX(1.5%)/ICG(2%) (FIG. 2D).

FIGS. 3A-3J illustrate absorbance spectrums of free and conjugated ICG. FIGS. 3A-3E illustrate absorbance at high concentration (100 μM) of free ICG (FIG. 3A), P/ICG (2%) (FIG. 3B), P/CTX(1.5%)/ICG(2%) (FIG. 3C), P/LLL(40%)/ICG(2%) (FIG. 3D), and P/LLL(40%)/CTX(1.5%)/ICG(2%) (FIG. 3E).

FIGS. 3F-3J illustrate absorbance at low concentration (3 μM) of free ICG (FIG. 3F), P/ICG(2%) (FIG. 3G), P/CTX(1.5%)/ICG(2%) (FIG. 3H), P/LLL(40%)/ICG(2%) (FIG. 3I), and P/LLL(40%)/CTX(1.5%)/ICG(2%) (FIG. 3J).

FIGS. 4A-4C illustrate fluorescent intensity and properties of imaging nanoagents. FIG. 4A illustrates fluorescence intensity of imaging nanoagents P/ICG(2%) (open square), P/CTX(1.5%)/ICG(2%) (closed square), P/LLL(40%)/ICG(2%) (open circle), P/LLL(40%)/CTX(1.5%)/ICG(2%) (closed circle) and control free ICG (asterisk) measured at pH 7.4. FIG. 4B is a schematic drawings of the imaging nanoagent P/CTX(1.5%)/ICG(2%) having the ICG molecules in close proximity to each other and demonstrating weak fluorescence.

FIG. 4C is a schematic drawing of the imaging nanoagent P/LLL(40%)/CTX(1.5%)/ICG(2%) having the ICG molecule interspaced by LLL away from each other and demonstrating high fluorescence.

FIGS. 5A-5B are photographs of tumor visualized by targeted imaging nanoagent P/LLL(40%)/CTX(1.5%)/ICG(2%) (FIG. 5A) and control imaging nanoagent P/LLL(40%)/ICG(2%) (FIG. 5B). The images at the top of the panels are marked “Visible”; the images in the middle of the panels are marked “NIR+Visible”; and the images at the bottom of the panels are marked “NIR”. FIG. 5A illustrates tumor visualization before incision (left panel, on the left), after small incision (left panel, on the right), after big incision (middle panel, on the left), after partial tumor resection (middle panel, on the right), and after complete tumor resection (right panel). FIG. 5B illustrates that control nanoagent failed to visualize tumors.

FIGS. 6A-6C illustrate pharmacokinetics measured as fluorescence intensity of the targeted imaging agents in serum and localization of the targeted and non-targeted imaging nanoagents (also referred to herein as nanodrugs). FIG. 6A illustrates serum fluorescence intensity for a targeted imaging nanoagent in serum. FIG. 6B illustrates concentration of the imaging nanoagent P/LLL(40%)/CTX(1.5%)/ICG(2%) in liver, kidney, heart, luna, spleen, tumor and normal brain. FIG. 6C illustrates concentration of the control non-targeted nanoagent P/LLL(40%)/ICG(2%) in the same organs as shown in FIG. 6B.

FIGS. 7A-7G illustrate accumulation of imaging nanoagents and contrast ratio between healthy brain and tumor area after administration of the nanoagents to a subject. FIGS. 7A-7F illustrate accumulation of the imaging nanoagents as function of time following administration. FIG. 7A illustrates accumulation of the imaging nanoagent and contrast ratio in brain tumor vs. surrounding healthy brain at 2 hours. FIG. 7B illustrates accumulation of the imaging nanoagent and contrast ratio at 4 hours. FIG. 7C illustrates accumulation of the imaging nanoagent and contrast ratio at 8 hours. FIG. 7D illustrates accumulation of the imaging nanoagent and contrast ratio at 12 hours. FIG. 7E illustrates accumulation of the imaging nanoagent and contrast ratio at 24 hours. FIG. 7F illustrates accumulation of the imaging nanoagent and contrast ratio at 48 hours. FIG. 7G illustrates accumulation of the imaging nanoagent in the tumor as function of time. Nanoagent was administered via I.V. tail vein injections.

FIG. 8 illustrates degradation of the targeted imaging nanoagent in human serum.

FIG. 9 illustrates imaging systems filter configurations: the use of very narrow band NIR Laser light to excite ICG at the wavelength of 785 nm aided by use of a Laser Cleanup filter to allow for maximum excitation efficiency, and in conjunction, with a Notch Filter in front of the camera to remove the excitation light from the image and capture only the fluorescence emission for the target.

FIGS. 10A-10D illustrate images of tumor and brain sections 16 hours after iv injection of imaging nanoagents containing rhodamine (Rh) into mouse tails of animals. FIG. 10A illustrates images of tumor and brain sections after injection of P/Rh(0.5%). FIG. 10B illustrates the brain section after injection of P/LLL(40%)/Rh(0.5%). FIG. 10C illustrates tumor and brain sections after injection of P/LLL(40%)/CTX(1.5%)/Rh(0.5%). FIG. 10D illustrates intense distribution of the imaging nanoagent P/LLL(40%)/CTX(1.5%)/Rh(0.5%) stained tumor cells and vessels along tumor margin. White dotted line represents tumor margin.

FIGS. 11A-11D illustrate binding of the imaging nanoagent (NIA) P/LLL(40%)/CTX(1.5%)/ICG(2%) and CTX/ICG to U87 MG glioma cells indicated by mean fluorescence intensity (MFI) of ICG measured by flow cytometry. FIG. 11A illustrates a flow cytometry histogram for binding of NIA as a function of concentration of total CTX, CTXtot. FIG. 11B illustrates a flow cytometry histogram for binding of CTX-ICG as function of total concentration of CTX. FIG. 11C illustrates a flow cytometry histogram for CTX competing with binding of NIA (content 5 μM CTXtot) at various concentrations of competing CTX (not fluorescent).

FIG. 11D illustrates a flow cytometry histogram for the mixture of CTX (125 μM) and P/LLL(40%) (12.5 μM), both not fluorescence labelled, competing with binding of NIA (content 5 μM CTXtot).

FIG. 12 illustrates binding of the imaging nanoagent P/LLL(40%)/CTX(1.5%)/Rh(0.5%) to glioma cells measured via mean fluorescence intensity (MFI) of Rh by flow cytometry.

FIG. 13 illustrates resection of tumor and evaluation of precision by microscopic inspection of H & E stained sections. An ex vivo H & E stained section is shown for measurement of the area. A region of interest (ROI) was drawn around tumor perimeter to determine total tumor volume. Similarly, ROI was drawn around leftover tumor area to determine remaining tumor. % resection is calculated in top, middle and deep tumor sections.

FIGS. 14A-14D illustrate U87 MG GBM xenografts after NIA-guided resection. Precision of tumor resection and interference with tumor infiltration. FIG. 14A, panel 1, illustrates, tumor slice (8 micron deep) containing the imaging nanoagent, P/LLL(40%)/CTX(1.5%)/ICG(2%), 4 h after i.v. injection of the NIA, visualized under Odyssey ELX; panel 2 illustrates magnification of tumor border to brain exhibiting interdigitation (arrows) into tumor-free tissue; panel 3, illustrates tumor H&E staining in border regions exhibiting tumor interdigitation into brain for comparison with panels 1 and 2. FIG. 14B, panels 1, 2, 3 illustrates the tumor fragment (infiltrating tumor cells) remaining after resection under NIR fluorescence of the injected imaging nanoagent. FIG. 14C, panels 1 and 2, illustrates brain resection under white light for estimation of resection precision. FIG. 14D illustrates efficiency of tumor resection under white light and NIR. H & E analysis was performed after section brain tissue in top, middle and deep areas. Quantification was performed after analyzing H& E sections to determine tumor volume.

### DETAILED DESCRIPTION OF THE PREFERRED EMBODIMENTS

Certain terminology is used in the following description for convenience only and is not limiting. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise.

The phrase “at least one” followed by a list of two or more items, such as “A, B, or C,” means any individual one of A, B or C as well as any combination thereof.

The words “right,” “left,” “top,” and “bottom” designate directions in the drawings to which reference is made.

Although methods and materials similar or equivalent to those described herein can be used in the practice or testing of this disclosure, suitable methods and materials are described below.

The terms “proliferative disorder” and “proliferative disease” refer to disorders associated with abnormal cell proliferation such as cancer.

The terms “tumor” and “neoplasm” as used herein refer to any mass of tissue that result from excessive cell growth or proliferation, either benign (noncancerous) or malignant (cancerous) including pre-cancerous lesions.

The terms “cancerous cell”, “tumor cell” and grammatical equivalents refer to a cell derived from a tumor or a pre-cancerous lesion including both a non-tumorigenic cell and a tumorigenic cell, i.e., cancer stem cell.

As used herein “tumorigenic” refers to the functional features of a solid tumor stem cell including the properties of self-renewal, i.e., giving rise to additional tumorigenic cancer cells, and proliferation to generate other tumor cells, i.e., giving rise to differentiated and thus non-tumorigenic tumor cells, such that cancer cells form a tumor.

The terms “subject” and “individual” are used interchangeably herein, and mean a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In an embodiment, the subject may be a mammal, e.g., a primate, e.g., a human. The terms, “patient” and “subject” are used interchangeably herein. The terms, “patient” and “subject” are used interchangeably herein.

Preferably, the subject is a mammal. The mammal may be a human, non-human primate, mouse, rat, dog, cat, horse, or cow, but are not limited to these examples. Mammals other than humans may be advantageously used as subjects that represent animal models of cancer. In addition, the methods described herein may be used to treat domesticated animals and/or pets. A subject may be male or female. A subject may be one who has been previously diagnosed with or identified as suffering from cancer, but need not have already undergone treatment.

An embodiment provides an imaging nanoagent comprising a polymalic acid-based molecular scaffold, a chlorotoxin peptide or a variant thereof, and at least one fluorescent moiety. The chlorotoxin peptide and the at least one fluorescent moiety may be covalently linked to the polymalic acid-based molecular scaffold.

As used herein, the term “polymalic acid” refers to a polymer, e.g., a homopolymer, a copolymer or a blockpolymer that contains a main chain ester linkage. The polymalic acid may be at least one of biodegradable and of a high molecular flexibility, soluble in water (when ionized) and organic solvents (in its acid form), non-toxic, or non-immunogenic (Lee B et al., Water-soluble aliphatic polyesters: poly(malic acid)s, in: Biopolymers, vol. 3a (Doi Y, Steinbuchel A eds., pp 75-103, Wiley-VCH, New York 2002, which is incorporated herein by reference as if fully set forth). In an embodiment, the polymalic acid may be poly(B-L-malic acid), herein referred to as poly-B-L-malic acid or PMLA. The polymalic acid may contain pendant carboxyl groups that may be linked to additional moieties.

Without limitations, the polymalic acid may be of any length and of any molecular mass. The polymalic acid may have a molecular mass of 5, 10, 20, 30, 40, 50, 60, 70, 80, 90, 95, or 100 kDa, or more. In an embodiment, the polymalic acid may have a molecular mass in a range between any two of the following molecular masses: 5, 10, 20, 30, 40, 50, 60, 70, 80, 90, 95, or 100 kDa.

Exemplary polymalic acid-based molecular scaffolds amenable to the imaging nanoagents disclosed herein are described, for example, in PCT Appl. Nos. PCT/US04/40660, filed Dec. 3, 2004, PCT/US09/40252, filed Apr. 10, 2009, and PCT/US10/59919, filed Dec. 10, 2010, PCT/US10/62515, filed Dec. 30, 2010; and U.S. patent application Ser. No. 10/580,999, filed Mar. 12, 2007, and Ser. No. 12/935,110, filed Sep. 28, 2010, contents of all which are incorporated herein by reference as if fully set forth.

The chlorotoxin peptide may be the native chlorotoxin (CTX) peptide. The native chlorotoxin is a 36 amino acid peptide isolated from the scorpion Leiurus quinquestriatus that selectively binds to cancerous cells. The native clorotoxin peptide may comprise, consists essentially of, or conisists of an amino acid sequence with at least 70, 72, 75, 80, 85, 90, 91, 92, 93, 94, 95, 96, 97, 98, 99 or 100% identity to SEQ ID NO: 1.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity is measured by the Smith Waterman algorithm (Smith T F, Waterman M S 1981 “Identification of Common Molecular Subsequences,” J Mol Biol 147: 195-197, which is incorporated herein by reference as if fully set forth).

The chlorotoxin peptide may be a variant of the native chlorotoxin peptide that retains some or all of the cancer-cell binding activity of chlorotoxin. The term “variant” refers to an amino acid sequence of a native chlorotoxin peptide having one or more amino acid residues substituted with an amino acid residue(s), which differ from the amino acid residue(s) of the native chlorotoxin in that position. The chlorotoxin peptide may be a variant of the chlotoxin peptide comprising the amino acid sequence of SEQ ID NO: 1. The native chlorotoxin peptide and the variants of the native chlorotoxin peptide are described in PCT Patent Application Publication Nos. WO2006115633 and WO2011142858, which are incorporated herein by reference as if fully set forth.

The chlorotoxin peptide may be a chlorotoxin-like peptide having some or all of the cancer-cell binding activity of a native chlorotoxin. The chlorotoxin-like peptides are described by Ali et al., “Structure-Activity Relationship of Chlorotoxin-Like Peptides,” Toxins, 2016, 8(2), 36, which is incorporated herein by reference as if fully set forth. The clorotoxin-like peptide may comprise, consists essentially of, or conisists of an amino acid sequence with at least 70, 72, 75, 80, 85, 90, 91, 92, 93, 94, 95, 96, 97, 98, 99 or 100% identity to the sequence selected from the group consisting of SEQ ID NOs: 2-10.

The polymalic acid based molecular scaffold may be a polymalic acid containing from 0.2% to 10% of pendant carboxylates (100%) conjugated to an amino acid residues of the chlorotoxin peptide. The polymalic acid may contain from 0.2% to 0.5%, from 0.5% to 1%, from 1% to 1.5%, from 1.5% to 2%, from 2% to 2.5%, from 2.5% to 3%, from 3% to 3.5%, from 3.5% to 4%, from 4% to 4.5%, from 4.5% to 5%, from 5% to 5.5%, from 5.5% to 6%, from 6% to 6.5%, from 6.5% to 7%, from 7% to 7.5%, from 7.5% to 8%, from 8% to 8.5%, from 8.5% to 9%, from 9% to 9.5%, or from 9.5% to 10% of pendant carboxylates conjugated to the amino acid residue(s) of the chlorotoxin peptide. The polymalic acid may contain 1.5% of pendant carboxylates conjugated to the amino acid residues of chlorotoxin peptides.

The fluorescent moiety may be any fluorescent reporter dye. A wide variety of fluorescent reporter dyes, e.g., fluorophores, are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters may include xanthene dyes, such as fluorescein or rhodamine dyes. Fluorophores may be, but are not limited to one or more of the following: 1,5 IAEDANS; 1,8-ANS; 4-Methylumbelliferone; 5-carboxy-2,7-dichlorofluorescein; 5-Carboxy fluorescein (5-FAM); 5-Carboxynapthofluorescein (pH 10); 5-Carboxytetramethyl rhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethyl rhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; or YOYO-3. Many suitable forms of these fluorescent compounds are available and may be used.

Examples of fluorescent proteins suitable for use as imaging agents include, but are not limited to one or more of the following: green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566, contents of which are incorporated herein by reference as if fully set forth). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, mOrange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

The fluorescent moiety may be one or more cyanine dyes. The cyanine dye may be but is not limited to indocyanine green (ICG), Cy5, Cy5.5, Cy5.18, Cy7 and Cy7.18, IRDye 78, IRDye 680, IRDye 750, IRDye 800 phosphoramidite, DY-681, DY-731, and DY-781.

The fluorescent moiety may be a fluorescent dye suitable for near-infrared (NIR) fluorescence. The NIR imaging may be used for intraoperative visualization and non-invasive imaging of cells and tissues in a subject. The NIR fluorescence imaging involves administration of a fluorescent contrast agent that can be excited at wavelengths of 780 nm or greater, and has a significant Stoke's shift emitting fluorescence at wavelengths of 800 nm or greater. The fluorescent dye used for NIR imaging may be ICG. The fluoresecent dye may be Rhodamine.

The polymalic acid based molecular scaffold may be a polymalic acid containing from 0.2% to 20% of pendant carboxylates (100%) conjugated to the fluorescent moieties. The polymalic acid may contain from 0.2% to 0.5%, from 0.5% to 1%, from 1% to 1.5%, from 1.5% to 2%, from 2% to 2.5%, from 2.5% to 3%, from 3% to 3.5%, from 3.5% to 4%, from 4% to 4.5%, from 4.5% to 5%, from 5% to 5.5%, from 5.5% to 6%, from 6% to 6.5%, from 6.5% to 7%, from 7% to 7.5%, from 7.5% to 8%, from 8% to 8.5%, from 8.5% to 9%, from 9% to 9.5%, from 9.5% to 10%, from 10% to 10.5%, from 10.5% to 11%, from 11% to 11.5%, from 11.5% to 12%, from 12% to 12.5%, from 12.5% to 13%, from 13% to 13.5%, from 13.5% to 14%, from 14% to 14.5%, from 14.5% to 15%, from 15% to 15.5%, from 15.5% to 16%, from 16% to 16.5%, from 16.5% to 17%, from 17% to 17.5%, from 17.5% to 18%, from 18% to 18.5%, from 18.5% to 19%, from 19% to 19.5%, or from 19.5% to 20% of pendant carboxylates conjugated to the fluorescent moieties. The polymalic acid may contain 2% of pendant carboxylates conjugated to the fluorescent moieties. The polymalic acid may contain 2% of pendant carboxylates conjugated to the ICG molecules. The polymalic acid may contain 0.5% of pendant carboxylates conjugated to the Rhodamine molecules.

In an embodiment, the imaging nanoagent may further comprise at least one biologically active molecular module.

As used herein “the biologically active molecular module” is a biologically active molecular structure ranging from a small drug molecule or chromophore molecule to a complete protein molecule such as an antibody or lectin. One or more biologically active molecular module may be an anti-cancer agent, a targeting ligand, or an endosomolytic ligand.

In an embodiment, the biologically active molecular module may be an anti-cancer agent. As used herein, the term “anti-cancer agent” refers to any compound (including its analogs, derivatives, prodrugs and pharmaceutical salts) or composition, which can be used to treat cancer. Anti-cancer agents may be, but are not limited to, inhibitors of topoisomerase I and II, alkylating agents, microtubule inhibitors or angiogenesis inhibitors.

The anti-cancer agent may be but is not limited to an antisense oligonucleotide, an siRNA oligonucleotide, an antibody, a polypeptide, an oligopeptide, a low molecular weight drug, radioisotope, toxin, cytotoxic agent, enzyme, sensitizing drug, nucleic acid, and anti-angiogenic agent.

Additional exemplary anti-cancer agents amenable to the present invention may be, but are not limited to: paclitaxel (taxol); docetaxel; germicitibine; aldesleukin; alemtuzumab; alitretinoin; allopurinol; altretamine; amifostine; anastrozole; arsenic trioxide; asparaginase; BCG live; bexarotene capsules; bexarotene gel; bleomycin; busulfan intravenous; busulfanoral; calusterone; capecitabine; platinate; carmustine; carmustine with polifeprosan implant; celecoxib; chlorambucil; cladribine; cyclophosphamide; cytarabine; cytarabine liposomal; dacarbazine; dactinomycin; actinomycin D; darbepoetin alfa; daunorubicin liposomal; daunorubicin, daunomycin; denileukin diftitox, dexrazoxane; docetaxel; doxorubicin; doxorubicin liposomal; dromostanolone propionate; Elliott's B solution; epirubicin; epoetin alfa estramustine; etoposide phosphate; etoposide (VP-16); exemestane; filgrastim; floxuridine (intraarterial); fludarabine; fluorouracil (5-FU); fulvestrant; gemtuzumab ozogamicin; goserelin acetate; hydroxyurea; ibritumomab tiuxetan; idarubicin; ifosfamide; imatinib mesylate; interferon alfa-2a; interferon alfa-2b; irinotecan; letrozole; leucovorin; levamisole; lomustine (CCNU); mechlorethamine (nitrogenmustard); megestrol acetate; melphalan (L-PAM); mercaptopurine (6-MP); mesna; methotrexate; methoxsalen; mitomycin C; mitotane; mitoxantrone; nandrolone phenpropionate; nofetumomab; LOddC; oprelvekin; pamidronate; pegademase; pegaspargase; pegfilgrastim; pentostatin; pipobroman; plicamycin; mithramycin; porfimer sodium; procarbazine; quinacrine; rasburicase; rituximab; sargramostim; streptozocin; talbuvidine (LDT); talc; tamoxifen; temozolomide; teniposide (VM-26); testolactone; thioguanine (6-TG); thiotepa; topotecan; toremifene; tositumomab; trastuzumab; tretinoin (ATRA); uracil mustard; valrubicin; valtorcitabine (monoval LDC); vinblastine; vinorelbine; zoledronate; or any mixtures thereof.

The imaging nanoagent may comprise at least two different anti-cancer agents covalently linked to the polymalic acid-based molecular scaffold.

In an embodiment, the biologically active molecular module may be a targeting ligand. As used herein the term “targeting ligand” refers to any molecule that provides an enhanced affinity for a selected target, e.g., a cell, cell type, tissue, organ, region of the body, or a compartment, e.g., a cellular, tissue or organ compartment. Targeting ligands may be, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL or HDL ligands.

In an embodiment, the targeting ligand may target a cancerous cell or tissue. As used herein, the phrase “target a cancerous cell or tissue” refers to delivery of an imaging nanoagent to a population of cancer-forming cells within tumors, i.e., cancerous cells or tissue.

In an embodiment, the targeting ligand may be an antibody specific to at least vasculature protein in a cell. In an embodiment, the vasculature protein may be a transferrin receptor protein. An antibody targeting module (TfR-Ab) may bind the transferrin receptor protein and thereby achieve transcytosis through endothelium associated with BBB. Without limitations, the antibody specific to the vasculature protein may be a monoclonal or polyclonal antibody. Further, the antibody may be a humanized antibody or a chimeric antibody.

The transferrin (Tf) receptor (TfR/CD71) is a transmembrane homodimer protein involved in iron uptake and cell growth regulation. Cancer cells express TfR at levels several-fold higher (up to 100-fold higher) than normal cells. TfR overexpression is correlated with stage and prognosis in various cancers, including breast cancer. High TfR expression levels on cancer cells, its ability to internalize, and its role in cancer pathology make it an attractive target for cancer therapy. Further, TfR has been used for delivery of a wide variety of cytotoxic molecules bound to Tf or anti-TfR mAbs by receptor-mediated endocytosis into different cancer cells including breast.

The blood-brain barrier is a high resistance barrier formed by tightly joined capillary endothelial cell membranes that maintains brain homeostasis and restricts brain access of multiple molecules including therapeutic Abs targeting cancer. However, BBB expresses TfR on its endothelial cells and anti-TfR mAbs can effectively cross BBB by transcytosis, a process used for brain delivery of therapeutic drugs including those targeting cancer. These in vitro, preclinical, and clinical studies show the efficacy and safety of targeting TfR to deliver therapeutic agents into cancer cells and are particularly relevant for drug delivery across BBB to treat deadly breast cancer brain metastases.

In an embodiment, the targeting ligand may be a lectin or another ligand specific to the transferrin receptor. In an embodiment, the targeting ligand may be a ligand to one of any number of cell surface receptors or antigens.

In an embodiment, the targeting ligand may be an antibody specific to EGFR, HER, or HER2/neu. In an embodiment, the anti-EGFR antibody mat be Cetuximab. In an embodiment, the anti-HER2/neu antibody may be Trastuzumab Herceptin®. It is noted that the anti-HER2/neu antibody or the anti-EGFR antibody may be a monoclonal or polyclonal antibody. Further, the anti-HER2/neu antibody or the anti-EGFR antibody may be a humanized antibody or a chimeric antibody.

The molecular scaffold and the components covalently linked with the polymalic acid-based molecular scaffold may be linked to each other via a linker. As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroaryl alkynyl, alkenylheteroarylalkyl, alkenylheteroarylalkenyl, alkenylheteroaryl alkynyl, alkynylheteroarylalkyl, alkynylheteroarylalkenyl, alkynylheteroaryl alkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In an embodiment, the linker may comprise a polyethylene glycol (PEG). Without limitations, the PEG may be of any desired molecular weight. In an embodiment, the PEG may have a molecular weight of about 250 Da, about 500 Da, about 1,000 Da, about 1,500 Da, about 2,000 Da, about 2,500 Da, about 3,000 Da, about 3,500 Da, about 4,000 Da, about 4,500 Da, about 5,000 Da, about 10,000 Da, about 15,000 Da, about 20,000 Da, about 25,000 Da, or about 30,000 Da. In an embodiment, the PEG may have a molecular weight of about 3,400 Da.

In an embodiment, the imaging nanoagent may further comprise a PK modulating ligand covalently linked with the polymalic acid-based molecular scaffold. As used herein, the terms “PK modulating ligand” and “PK modulator” refers to molecules which can modulate the pharmacokinetics of the imaging nanoagent. For example, the PK modulator can inhibit or reduce resorption of the imaging nanoagent by the reticuloendothelial system (RES) and/or enzyme degradation.

PEGylation is generally used in drug design to increase the in vivo half-life of conjugated proteins, to prolong the circulation time, and enhance extravasation into targeted solid tumors (Arpicco et al., 2002 Bioconjugate Chem 13:757 and Maruyama et al., 1997 FEBS Letters 413:1771, which is incorporated herein by reference as if fully set forth). Thus, in an embodiment, the PK modulator may be a PEG. Without limitations, the PEG may be of any desired molecular weight. In an embodiment, the PEG may have a molecular weight of about 1,000 Da, about 1,500 Da, about 1,000 Da, about 2,500 Da, about 3,000 Da, about 3,500 Da, about 4,000 Da, about 4,500 Da, about 5,000 Da, about 10,000 Da, about 15,000 Da, about 20,000 Da, about 25,000 Da, or about 30,000 Da. In an embodiment, the PK modulator may be PEG of about 5,000 Da. Other molecules known to increase half-life may also be used as PK modulators.

In an embodiment, the biologically active molecular module may be an endosomolytic ligand. The endosomolytic ligand may be covalently linked with the polymalic acid-based molecular scaffold. As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. The endosomolytic ligands may be, but are not limited to, imidazoles, poly or oligoimidazoles, linear or branched polyethyleneimines (PEIs), linear or branched polyamines, e.g. spermine, cationic linear or branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural or synthetic fusogenic lipids, natural or synthetic cationic lipids.

In an embodiment, the endosomolytic ligand may include a plurality of leucine or valine residues. The endosomolytic ligand may be polyleucine. In an embodiment, endosomolytic ligand may be Leu-Leu-Leu (LLL).

The polymalic acid-based molecular scaffold may be a polymalic acid containing from 20% to 70% of pendant carboxylates (100%) conjugated by amide bond involving the N-terminal —NH₂— of oligopeptide trileucine LLL

The polymalic acid may contain from 20% to 25%, from 25% to 30%, from 30% to 35%, from 35% to 40%, from 40% to 45%, from 45% to 50%, from 50% to 55%, from 55% to 60%, gtom 60% to 65% or from 65% to 70% of pendant carboxylates conjugated to the oligopeptide LLL. The polymalic acid may contain 40% of pendant carboxylates conjugated to the oligopeptide LLL.

In an embodiment, the imaging nanoagent may contain two or more fluorescent moieties conjugated to polymalic acid-based molecular scaffold and interspaced with the trileucine (LLL) oligopeptide. For example, the trileucine (LLL) oligopeptide conjugated to the polymalic acid may be positioned in-between each two fluorescent moieties conjugated to the same polymalic acid-based molecular scaffold. The interspacing of the fluorescent moieties with LLL may prevent self-quenching of the fluorescent moieties and increase the intensity of fluorescence of the imaging nanoagent. In an embodiment, the the imaging nanoagent may comprise ICG molecules interspaced with trilleucine oligopeptides, and clorotoxin peptides. In a non-limiting example, the polymalic based molecular scaffold of the imaging nanoagent may comprise 2% of pendant carboxylates conjugated to the IGC molecules, 1.5% chlorotoxin peptides and 40% of the tri-leucine (LLL) oligopeptides. The exemplary imaging nanoagent may have ICG molecules interspaced with LLL oligopeptides.

Without limitations, the imaging nanoagent may be of any desired size. For example, the imaging nanoagent may be of a size that allows the imaging nanoagent to cross the blood-brain barrier via transcytosis. In an embodiment, the imaging nanoagent may range in size from about 1 nm to about 100 nm; from about 1 nm to about 10 nm; from about 10 nm to about 20 nm; from about 20 nm to about 30 nm; from about 30 nm to about 40 nm; from about 40 nm to about 50 nm; from about 50 nm to about 60 nm; from about 60 nm to about 70 nm; from about 70 nm to about 80 nm; from about 80 nm to about 90 nm; from about 90 nm to about 100 nm; from about 5 nm to about 90 nm; from about 10 nm to about 85 nm; from about 20 nm to about 80 nm; from about 25 nm to about 75 nm. In an embodiment, the imaging nanoagent may be about 50 nm to about 70 nm in size. In an embodiment, the imaging nanoagent may be 50 nm or less in size.

It will be understood by one of ordinary skill in the art that the imaging nanoagent may exhibit a distribution of sizes around the indicated “size.” Thus, unless otherwise stated, the term “size” as used herein refers to the mode of a size distribution of imaging nanoagents, i.e., the value that occurs most frequently in the size distribution. Methods for measuring the size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

Without limitations, the imaging nanoagent may be of any desired molecular weight. In an embodiment, the molecular weight of the imaging nanoagent may range from about from about 5 kDa to about 10 kDa, from about 10 kDa to about 20 kDa, from about 20 kDa to about 30 kDa, from about 30 kDa to about 40 Da, from about 40 kDa to about 50 kDa, from about 50 kDa to about 60 kDa, from about 60 kDa to about 70 kDa, from about 70 kDa to about 80 kDa, from about 80 kDa to about 90 kDa, from about 90 kDa to about 100 kDa, from about 100 kDa to about 105 kDa, from about 105 kDa to about 110 kDa, from about 110 kDa to about 120 kDa, from about 120 kDa to about 130 kDa, from about 130 kDa to about 140 Da, from about 140 kDa to about 150 kDa, from about 150 kDa to about 160 kDa, from about 160 kDa to about 170 kDa, from about 170 kDa to about 180 kDa, from about 180 kDa to about 190 kDa, from about 190 kDa to about 200 kDa, from about 200 kDa to about 300 kDa, from about 300 kDa to about 400 kDa, from about 400 kDa to about 500 kDa, from about 500 kDa to about 600 kDa, from about 600 kDa to about 700 kDa, from about 700 kDa to about 800 kDa, from about 800 kDa to about 900 kDa, from about 900 kDa to about 1000 kDa, from about 1000 kDa to about 1100 kDa, from about 1100 kDa to about 1200 kDa, from about 1200 kDa to about 1300 kDa, from about 1300 kDa to about 1400 kDa, from about 1400 kDa to about 1500 kDa, from about 1500 kDa to about 1600 kDa, from about 1600 kDa to about 1700 kDa, from about 1700 kDa to about 1800 kDa, from about 1800 kDa to about 1900 kDa, or from about 1900 kDa to about 2000 kDa.

In an embodiment, the molecular weight of the imaging nanoagent may be about 5 kDa to about 200 kDa. In an embodiment, the molecular weight the imaging nanoagent may be about 192 kDa.

In an embodiment, a pharmaceutically acceptable composition comprising any one the imaging nanoagents disclosed herein and a pharmaceutically acceptable carrier or excipient is provided.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zincstearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (S) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyllaurate; (13) agar; (14) buffering agents, such as magnesium hydroxide and aluminum hydroxide; (15) alginic acid; (16) pyrogen-free water; (17) isotonic saline; (IS) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the likes are used interchangeably herein.

In an embodiment, a method for detecting and removing a cancer is provided. The method may include administering any one of the imaging nanoagents described herein or any one of the pharmaceutically acceptable compositions described herein to a subject to detect cancerous cells or tissue.

The imaging nanoagent may be administered to the subject from 2 to 60 hours prior to the surgery. The imaging nanoagent may be administered 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, 55 hours, 60 hours, or at any time in between any two values set forth herein prior to the surgery.

As used herein, the term “administer” refers to the placement of a composition into a subject by a method or route which results in at least partial localization of the composition at a desired site such that desired effect is produced. A compound or composition described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration.

Exemplary modes of administration include, but are not limited to, injection, infusion, instillation, inhalation, or ingestion. “Injection” include, without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebro spinal, and intrastemal injection and infusion. In an embodiment, the compositions may be administered by intravenous infusion or injection.

The method may further comprise detecting the presence or absence of the imaging nanoagent. The presence of the imaging nanoagent in the cells or tissues may indicate the presence of cancerous cells or tissue. The step of detecting may be performed by an imaging technique. The imaging may be an optical imaging technique such as, for example, near infrared (NIR) imaging. The NIR imaging involves excitation of a fluorophore that emits light at a wavelength in the red or far red end of the light spectrum (longer than 600 nm). Equipment suitable for optical imaging is well-known in the art and generally consists of a light source, filters, detector, and appropriate electronics for signal processing as described in Kittle et al., 2014 “Fluorescence-Guided Tumor Visualization Using the Tumor Paint BLZ-100”, Cureus 6:e210, and Butte et al., 2014 “Near-infrred imaging of brain tumors using the tumor Pain BLZ-100 to Achieve Near-complete Resection of Brain Tumors, Nuerosurgical focus, 36(2), E1, both of which are incorporated herein by reference as if fully set forth. In a non-limiting example, the presence of cancerous cells or tissues following administration of the imaging nanoagent may be visualized by using the Synchronized Near-InfraRed Imaging System (SIRIS) described in these references. The cancerous cells or tissues may be visualized by any other device capable of detecting fluorescence of the fluorescent moiety, for example, ICG. The step of detection may include acquiring an image or images of the cancerous cells or tissues in the subject. For example, the SIRIS may acquire a first image under white light mode and a second image under near-infrared fluorescence, and superimpose these images on a high definition (HD) video monitor. The first and the second images may be acquired simultaneously. The cancerous or tumorigenic cells accumulating the imaging nanoagent may fluoresce and produce a visible border of the tumor on the monitor of the device during surgery. Once the fluorescing borders of the tumor, or fluorescing cancerous cells are identified, the method may further comprise surgically removing the cancerous cell or tissue of the tumor. The method may comprise removing from 90% to 99.8% of the cancerous cells following resection of the tumor.

As used herein, the term “cancer” refers to an uncontrolled growth of cells that may interfere with the normal functioning of the bodily organs and systems. The cancer may be either a primary cancer, or a metastatic cancer, or both. Cancers that migrate from their original location and seed vital organs can eventually lead to the death of the subject through the functional deterioration of the affected organs. Metastasis is a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination.

As used herein, the term “cancer” also includes, but is not limited to, solid tumors and blood born tumors. The term cancer refers to disease of skin, tissues, organs, bone, cartilage, blood and vessels. The term “cancer” further encompasses primary and metastatic cancers. Examples of cancers that can be treated with the method of the invention include, but are not limited to solid tumors; brain cancer, including but not limited to gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas; breast cancer, including but not limited to ductal carcinoma in situ, invasive (or infiltrating) ductal carcinoma, invasive (or infiltrating) lobular carcinoma, adenoid cystic (or adenocystic) carcinoma, low-grade adenosquamous carcinoma, medullary carcinoma, mucinous (or colloid) carcinoma papillary carcinoma, tubular carcinoma, inflammatory breast cancer, Paget disease of the nipple, phyllodes tumor, triple negative breast cancer, metastatic breast cancer; carcinoma, including that of the bladder, breast, colon, kidney, lung, ovary, pancreas, stomach, cervix, thyroid, and skin, including squamous cell carcinoma; other tumors including melanoma, seminoma, tetratocarcinoma; tumors of the central and peripheral nervous system; and other tumors including, but not limited to, xenoderma, pigmentosum, keratoactanthoma, thyroid follicular cancer, and teratocarcinoma.

In an embodiment, a method of imaging cells or tissue in a brain of a subject is provided. The method may comprise administering any one of the imaging nanoagents disclosed herein or ant one of the pharmaceutically acceptable compositions described herein to a subject in need thereof. The method may further comprise visualizing the imaging nanoagent. The step of the visualizing may be performed by the NIR imaging. The step of visualizing may be performed in vivo.

In an embodiment, a method for treating cancer in a subject is provided. The method may comprise administering any one of the imaging nanoagents or any one of the pharmaceutically acceptable composition described herein to a subject in need thereof. The cancer may be a primary cancer, a metastatic cancer, or both.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The method may further comprise performing a surgery to remove a cancer detected by the imaging agent.

In an embodiment, the method may further comprise co-administering an additional therapeutic agent to the subject.

As used herein, the term “co-administering,” “co-administration,” or “co-administer” refers to the administration of at least two different compounds and/or compositions, wherein the compounds and/or the compositions may be administered simultaneously, or at different times, as long as they work additively or synergistically to treat cancer. Without limitations, the two different compounds and/or compositions may be administered in the same formulation or in separate formulations. When administered in separate formulations, the compounds and/or compositions may be administered within any time of each other. For example, the compounds and/or compositions may be administered within 24 hours, 12 hours, 6 hours, 5 hours, 4 hours, 3 hours, 2 hours, 1 hour, 45 minutes, 30 minute, 25 minutes, 20 minutes, 15 minutes, 10 minutes, 5 minutes or less of each other. Further, when administered in separate formulations, the compounds and/or compositions may be administered in any order. Additionally, co-administration does not require that the co-administered compounds and/or compositions be administered by the same route. As such, each may be administered independently or as a common dosage form. Further, the two compounds may be administered in any ratio to each other by weight or moles. For example, two compounds may be administered in a ratio of from about 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:25, 1:30, 1:40, or 1:50. The ratio may be based on the effective amount of either compound.

The additional therapeutic agent may be selected from the group consisting of: an antibody, an enzyme inhibitor, an antibacterial agent, an antiviral agent, a steroid, a non-steroid-inflammatory agent, an antimetabolite, a cytokine, a cytokine blocking agent, an adhesion molecule blocking agent, and a soluble cytokine receptor.

In an embodiment, the method may further comprise co-administering one or more additional anti-cancer therapy to the patient. In an embodiment, the additional therapy may be selected from the group consisting of chemotherapy, radiation therapy, thermotherapy, immunotherapy, hormone therapy, laser therapy, anti-angiogenic therapy, and any combinations thereof. In an embodiment, the additional therapy may comprise administering an anti-cancer agent to the patient.

In an embodiment, the method may comprise co-administering the imaging nanoagent and an anti-cancer agent or chemotherapeutic agent to the subject.

In an embodiment, the method may comprise co-administering an antineoplastic agent. The antineoplastic agents may include agents for overcoming trastuzumab resistance. A variety of agents including monoclonal antibodies, recombinant proteins, and drugs, are known to have activity in treating breast cancer, and are here contemplated to be useful agents in combination with compositions described herein.

In an embodiment, the method may include co-administering paclitaxel (taxol, Bristol-Myers Squibb); docetaxel (taxotere, Sanofi-Aventis); dasatinib, (Sprycel®, Bristol-Myers Squibb) a small-molecule tyrosine kinase inhibitor; gefitinib (Iressa, Astra Zeneca and Teva), an EGFR inhibitor; trastuzumab; an agent that decreases levels of phosphorylated HER2 and phosphorylated HEM; an agent that induces caspase-independent apoptosis as determined by the lack of an effect of caspase inhibitors on apoptosis; an agent that affects DNA repair machinery and leads to accumulation of double-stranded breaks (DSBs); erlotinib (Tarceva, Roche), an inhibitor of EGFR; an agent that affects a transcription factor associated with Williams-Beuren syndrome (WSTF, also known as BAZIB), a tyrosine kinase component of the WICH complex (WSTF-ISWI ATP-dependent chromatin-remodeling complex), that regulates the DNA damage response through phosphorylation of Tyr142 of H2AX; lapatinib (Tyverb®, GSK), a dual EGFR/HER2 tyrosine kinase inhibitor; pertuzumab (2c4, omnitarg, Genentech), a monoclonal antibody specific for the extracellular domain of HER2 protein; trastuzumab-DM1 comprised of trastuzumab and DM1, an agent that is an inhibitor of tubulin polymerization derived from maytansine; a PI3K pathway inhibitor; HER2 vaccines and adoptive immunotherapy targeting the HER2 extracellular domain; ertumaxomab (Rexomum, Fresenius Biotech GmbH), a bispecific antibody targeting HER2 and CD3 on T cells; defucosylated trastuzumab; or any combinations thereof.

In an embodiment, the method may comprise administering a therapeutically effective amount of any one of the imaging nanoagents or additional therapeutic agents described herein to a subject in need thereof.

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. In connection with treating cancer, the “therapeutically effective amount” is that amount effective for preventing further development of a cancer or transformed growth, and even to effect regression of the cancer or solid tumor.

Determination of a therapeutically effective amount is generally well within the capability of those skilled in the art. Generally, a therapeutically effective amount can vary with the subject's history, age, condition, sex, as well as the severity and type of the medical condition in the subject, and administration of other agents alleviate the disease or disorder to be treated.

Toxicity and therapeutic efficacy may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. As used herein, the term ED denotes effective dose and is used in connection with animal models. The term EC denotes effective concentration and is used in connection with in vitro models.

The data obtained from the cell culture assays and animal studies may be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose may be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

The dosage may be determined by a physician and adjusted, as necessary, to suit observed effects of the treatment. Generally, the compositions may be administered so that the active agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In an embodiment, the compositions may be administered at a dosage so that the active agent has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule may vary from once a week to daily depending on a number of clinical factors, such as the subject's sensitivity to the polypeptides. The desired dose may be administered every day or every third, fourth, fifth, or sixth day. The desired dose may be administered at one time or divided into subdoses, e.g., 2-4 subdoses and administered over a period of time, e.g., at appropriate intervals through the day or other appropriate schedule. Such sub-doses may be administered as unit dosage forms. In an embodiment, administration may be chronic, e.g., one or more doses daily over a period of weeks or months. Examples of dosing schedules may include administration daily, twice daily, three times daily or four or more times daily over a period of 1 week, 2 weeks, 3 weeks, 4 weeks, 1 month, 2 months, 3 months, 4 months, 5 months, or 6 months or more.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1—Fluorescent Guided Resection of Tumors

Tumor-Specific Targeting of Glioma with Chlorotoxin.

Cholorotoxin (CTX) is a 36-amino acid knottin peptide that avidly binds to many human malignancies including glioma while demonstrating essentially no binding to normal human tissues (reviewed in Stroud et al., Curr Pharm Des. 2011; 17:4362-4371; Wu et al., Chinese Journal of Cancer. 2010; 29:626-630; Mamelak and Jacoby, Expert Opin Drug Deliv. 2007; 4:175-186, which are incorporated herein by reference as if fully set forth). Annexin A2 complex is the likely receptor for CTX binding. Annexin A2 is not expressed on the cell surface of normal mammalian tissues other than human umbilical vein endothelial cells, but is highly expressed on the cell surface of several malignancies such as glioma (Kesavan et al., J Biol Chem. 2010; 285:4366-4374, which is inorporated herein by reference as if fully set forth). Once bound to the cell surface, CTX is internalized. This property, along with its small size and lack of immunogenicity, make CTX attractive as a ligand for targeted cancer therapies (Stroud et al., Curr Pharm Des. 2011; 17:4362-4371; Wu et al., Chinese Journal of Cancer. 2010; 29:626-630; Mamelak and Jacoby, Expert Opin Drug Deliv. 2007; 4:175-186; Kesavan et al., J Biol Chem. 2010; 285:4366-4374; Dardevet et al., Toxins 2015:7:1079-1101; Hockaday et al., The Journal of Nuclear Medicine. 2005; 46:580-586; 23. Mamelak et al., Journal of Clinical Oncology. 2006; 24:3644-3650; Gribbin et al., J Clin Oncol. 2009; 27 (suppl):abstr e14507; Butte et al., Neurosurg Focus. 2014; 36:E1; Veiseh et al., Cancer Res. 2007; 67:6882-6888; Huang et al., Clin Cancer Res. 2012; 18:5731-5740; Lyons et al., Glia. 2002; 39:162-73; and Soroceanu et al. Cancer Res. 1998; 58:4871-4879; all of which are inorporated herein by reference as if fully set forth).

Use of polymalic acid (PMLA) scaffold. The concept of a nanoagent that can target glioma cells for intraoperative fluorescence-guided resection (FGR) is extremely appealing. PMLA is suited to provide scaffold for covalent attachment of CTX and ICG. In this setting a nanoconjugate containing both CTX and ICG is used to target tumor cells for intraoperative FGR. A major appeal of this strategy is that the number of CTX and ICG moieties on the PMLA scaffold can be optimized to maximize tumor binding and/or fluorescence detection properties while minimizing dose requirements and potential toxicity. Further, PMLA has a very favorable toxicity profile making it well suited for further development as a clinical nanodrug (Ljubimova et al., J Drug Target. 2013; 21:956-967; Ljubimova et al., 2014, J Vis Exp: (88), which is inorporated herein by reference as if fully set forth). Various moieties can be attached to PMLA including antibodies, peptides, oligonucleotides, contrast agents and chemotherapeutics to optimize this molecule for pre-clinical and clinical use (Lee et al., Bioconjug. Chem. 2006; 17:317-326; Ding et al., Proc Natl Acad Sci USA. 2010; 107:18143-18148; Inoue et al., PLoS One. 2012; 7:e31070; Patil et al., ACS Nano, 2015; 9:5594-5608; Ding et al., Nanomedicine: Nanotechnology, Biology, and Medicine 2017; 13:631-639; Patil et al., (2015) Macromol Biosci. 15:1212-1217; Patil et al., Pharm Res. 2010; 27: 2317-29; Patil et al., Int. J. Mol. Sci 2012; 13:11681-93 (2012) (Special issue: Bioactive Nanoparticles), which are inorporated herein by reference as if fully set forth). The diverse functionality of PMLA nanoconjugates can be utilized to its fullest potential to optimize the ratio of tumor ligand to fluorescent marker, providing a transformative and quantum leap forward in the development of tumor-specific fluorescence technologies. Further, success in this arena with gliomas will undoubtedly lead to its widespread application in other surgical settings such as lung, head and neck cancers, melanoma and other solid tumors.

Nanotechnology for the engineering of drug delivery devices for cancer treatment. One of the primary advantages of a nano drug delivery vehicles containing PMLA is their ability to cross membrane barriers, particularly in the central nervous system (CNS) (Ding et al., Proc Natl Acad Sci USA. 2010; 107:18143-18148; Patil et al., ACS Nano, 2015; 9:5594-5608, which are inorporated herein by reference as if fully set forth).

This is important for drug delivery through blood-brain barrier (BBB). PMLA platform was chosen for its attractive properties as a drug carrier because of high loading capacity, biodegradability, stability in the bloodstream, ready cellular uptake, deep tissue penetration, and lack of toxicity and immunogenicity (Ljubimova et al., J Drug Target. 2013; 21:956-967; Ljubimova et al., 2014, J Vis Exu: (88)). PMLA-based imaging agents and drugs are of nano size (20-30 nm) and have special moieties for (1) endothelial transcytosis, (2) antisense oligonucleotides (AONs) inhibiting biosynthesis of a specific tumor marker, (3) peptides or tumor-specific mAbs for receptor-mediated endocytosis, (4) non-toxic pH-dependent trileucine moiety for the nanocarrier escape from endosomes by membrane disruption, (5) PEG for stability, and features for releasing the drug from the carrier. Besides, radioactive or MRI/fluorescent tracer can be conjugated to PMLA to follow drug distribution in biological fluids (blood, urine, spinal fluid), tissues and cells (Ding et al, Nanomedicine: Nanotechnology, Biology, and Medicine 2017; 13:631-639, which is inorporated herein by reference as if fully set forth). Importantly, molecules for multiple targeting can be easily and covalently attached to one PMLA molecule. Technical possibilities for reproducible syntheses, toxicity evaluation and efficacy of treatment of the multifunctional nanopolymers are medical reality now and are no longer scientific “nano” fiction (Bertrand et al., Adv Drug Deliv Rev. 2014; 66:2-25). Compared to existing nanomedicines, experimental and already used in clinic (Doxil, Abraxane, etc.), nanobioconjugates have several significant advantages, especially for brain tumor treatment: they can pass through BBB not by slow and inefficient EPR effect, but by active transcytosis through tumor vasculature without losing their payload (Ding et al., Proc Natl Acad Sci USA. 2010; 107:18143-18148; Patil et al., ACS Nano, 2015; 9:5594-5608, which are inorporated herein by reference as if fully set forth). Covalent binding of all moieties to the polymeric nanoplatform ensures delivery to the tumor site without leakage common to nanoparticles and liposomes. Dual targeting of tumor vasculature and cancer cells ensures specific drug delivery to its intended target without appreciable effect on adjacent normal tissues. They are fully biodegradable and non-toxic in animals. These significant advantages make polymeric nanoconjugates very attractive drugs for translational applications to treat brain cancer.

The imaging nanoagents described herein advantageously combine the tumor-specific binding properties of CTX, the utility of the NIR fluorescent Indocyanine Green (ICG) dye, and polymalic acid-based nanoconjugates. The use of a PMLA backbone allows for covalent attachment of chlorotoxin (CTX) for targeting and internalization into brain tumor cells and combining NIR fluorophore (ICG) with intense fluorescence for deep tissue penetration. The functional groups are either FDA approved or have low/negligible toxicity. The nano agent owning these integrated functions will have greater fluorescence detection capabilities.

### Example 2—Materials and Methods

Reagents. Highly purified, poly(B-L-malic acid), was prepared from the culture broth of Physarum polycephalum as described (Ljubimova et al., (2014) J Vis Exp; (88)). CTX was purchased form Bachem Americas (Torrance Calif., USA) Inc. Maleimide-PEG20000-SCM (MAL-PEG 2000-SCM) was obtained from Laysan Bio Inc. (Arab Al, USA). 3-(2-Pyridyldithio)-propionate (PDP) was synthesized as described³⁴. ICG-MAL was obtained from Intrace Medical, Lausanne, Switzerland. Unless otherwise indicated, all chemicals and solvents of highest purity were purchased from Sigma-Aldrich (St. Louis Mo., USA).

Analytical methods for synthesis of ICG-PMLA conjugates. The conjugation reaction of 2-MEA with PMLA was followed by thin layer chromatography (TLC) on precoated silica gel 60 F254 aluminum sheets (Sigma-Aldrich, St. Louis Mo., USA) and visualization of spots by UV light and by ninhydrin staining. Size exclusion chromatography was performed on an Elite LaChrom analytical system with Diode Array Detector L 2455 (Hitachi) and MW was measured using PolySep-GFC-P 4000 (300×7.80 mm) (Phenomenex) with PBS as a mobile phase and polystyrene sulfonates of known molecular weight as standards. Thiol residues attached to PMLA were assayed by the method of Ellman's reagent. Content of CTX in nanoconjugates was determined by Pierce™ BCA Protein Assay Kit (Thermo Scientific, Canoga Park, Calif.). Known amounts of Free CTX were used as standards. Quantification of malic acid in nanoconjugates was performed by the malate dehydrogenase assay after acid hydrolysis (Ding et al., Int J Mol Sci. 2015; 16:8607-8620). Percentage (%) of the nanoconjugate loading with CTX and ICG was calculated by using the formula %=100×(gmol ligand)/(gmol malic acid).

Synthesis of Preconjugate.

N-Hydroxysuccinimide (NHS; 0.62 mmol) and N,N′-dicyclohexylcarbodiimide (DCC; 1 mmol) dissolved in 2 ml of dimethyl formamide (DMF) were added consecutively to the solution of 36 mg of PMLA (0.31 mmol with regard to malyl units) dissolved in 0.7 ml of anhydrous acetone under vigorous stirring at RT. After stirring at RT for 2 hrs to complete the activation of carboxyl groups, MEA (0.05 mmol in DMF; 100 μl, 5 Mol-% with regard to malyl units) was added to the reaction mixture followed by equivalent amount of triethylamine (TEA) and reaction mixture was stirred at RT for 45 min. A solution of phosphate buffer (100 mM sodium phosphate and 150 mM NaCl, pH 6.8) was added at a ratio of 1:3 (organic solvent: buffer) and the reaction mixture was stirred at RT for 1 h. After centrifugation at 1,500×g for 10 min the clear supernatant was passed over a Sephadex PD-10 columns (GE Healthcare Waltham, Mass. USA) pre-equilibrated with deionized (DI) water. The product containing fractions were collected and freeze dried.

Synthesis of CTX-PEG2000-MAL.

A solution of CTX (1 mg, 0.26 micro mol) dissolved in 0.2 ml of sodium borate buffer (0.15 M, 0.1 mM EDTA, pH 8.0) was added to MAL-PEG2000-NHS (1.63 mg, 0.78 micro mol) dissolved in 0.2 ml of DMF. Reaction mixture was stirred at ambient temperature for 1 h. Then 0.4 ml of phosphate buffer (100 mM, pH 6.3) was added and the solution was passed over PD-10. The eluting fractions containing the product were directly used for conjugation to preconjugate.

Synthesis of P/ICG(2%).

To a solution of Preconjugate at 4 mg/ml dissolved in buffer (100 mM sodium phosphate, pH 5.5) was added a solution of ICG-Mal prepared as 2 mg/ml in DMF. Reaction mixture was stirred at RT for 1 h. Leftover thiol groups were blocked by the reaction with pyridyldithiopropionate (PDP). The reaction mixture was purified over PD-10 column in PBS, passed through 0.2-micron pore filters, and stored at −20° C.

Synthesis of P/CTX(1.5%)/ICG(2%). A solution of CTX-PEG2000-MAL, 4 mg/ml dissolved in buffer (100 mM sodium phosphate, pH 6.3) was dropwise added to 4 mg/ml of preconjugate at RT in the same buffer. The reaction was monitored by SEC-HPLC. After reaction completion (30 min), the pH of the reaction mixture was adjusted to 5.5 with 1 M citrate buffer, and 2 mg/ml of ICG-MAL in DMF was added. After reaction completion (remaining free SH groups were blocked with PDP³¹. The obtained imaging agent P/CTX(1.5%)/ICG(2%) was purified over PD-10 column in PBS, passed through 0.2-micron pore filters, snap-frozen and stored at −20° C.

Synthesis of P/LLL(40%)/ICG(2%).

To a solution of Preconjugate P/LLL(40%)/MEA(10%) at 4 mg/ml dissolved in buffer (100 mM sodium phosphate, pH 5.5) was added a solution of ICG-MAL prepared as 2 mg/ml in DMF. Reaction mixture was stirred at RT for 1 h. Leftover thiol groups were blocked by the reaction with pyridyldithiopropionate (PDP). The reaction mixture was purified over PD-10 column in PBS, passed through 0.2-micron pore filters, and stored at −20° C.

Synthesis of P/LLL(40%)/CTX(1.5%)/ICG(2%).

A solution of CTX-PEG2000-MAL, 4 mg/ml dissolved in buffer (100 mM sodium phosphate, pH 6.3) was dropwise added to 4 mg/ml of preconjugate P/LLL(40%)/MEA(10%) at RT in the same buffer. The reaction was monitored by SEC-HPLC. After reaction completion (30 min), the pH of the reaction mixture was adjusted to 5.5 with 1 M citrate buffer, and 2 mg/ml of ICG-MAL in DMF was added. After reaction completion (remaining free SH groups were blocked with PDP³¹. The obtained imaging agent P/LLL(40%)/CTX(1.5%)/ICG(2%) was purified over PD-10 column in PBS, passed through 0.2-micron pore filters, snap-frozen and stored at −20° C.

Hydrodynamic Diameter and Zeta Potential.

Synthesized conjugates were characterized with respect to their size and potential using Zetasizer Nano ZS90 (Malvern Instruments, United Kingdom). For the particle size measurements at 25° C., the solutions were prepared in PBS at a concentration of 2 mg/ml. For the measurement of the ζ potential, the concentration of the sample dissolved in 10 mM NaCl solution was 2 mg/ml, and the voltage applied was 150 mV. Data represent the mean of three measurements±standard deviation.

Cell lines and culture conditions.

Primary glioblastoma U87MG cell line was a gift from Drs. Webster Cavenee and Frank Furnari (UC San Diego), and cultured in minimum essential medium (MEM) supplemented with 10% fetal bovine serum (FBS), 1% MEM non-essential amino acids, 1 mM sodium pyruvate and 2 mM L-glutamine. MDA-MB-468 was cultured in Leibovitz's L-15 medium with 10% FBS at 37° C. without CO₂.

Tumor Xenografts and Nanoagent Treatment.

All experiments with animals were performed in strict accordance with the protocols approved by the Cedars-Sinai Medical Center Institutional Animal Care and Use Committee (IACUC). Athymic NCr-nu/nu female mice were obtained from NCI-Frederick. Human U87MG GBM cells were stereotactically implanted at mounts of 2.5×10⁴ cells into the left basal ganglia. Animals were monitored regularly for any potential symptoms. On day 20-25, at which point the tumor will have reached an average size of 2-4 mm in diameter animals were intravenously injected via tail vein with nanoagent's and imaged by the SIRIS imaging system for drug uptake in tumors and vital organs.

Tumor Visualization Device for Clinical Intra-Operative Imaging.

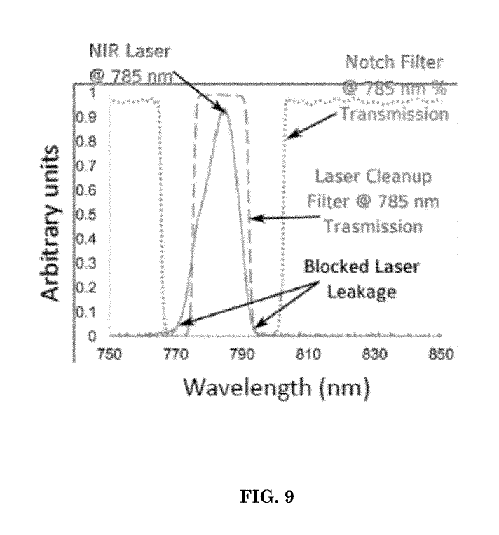

A device for intra-operative detection of NIR fluorescence has been built for clinical use in the resection of brain tumor. It simultaneously acquires and superimposes both white light (WL) and NIR images on a high definition (HD) video monitor. The system uses a single camera typical for endoscope-based systems applicable in surgical visualization. The device for illumination and sensing consists of a dual high definition charge coupled (CCD) camera that splits incident light into two pathways, one for white light and the second for NIR light (AD-130GE, ⅓″, 1296X966, 31 fps, GigE). NIR excitation is provided via a narrow band 785 nm laser diode (Thorlabs) at the peak ICG absorption. Excitation light is excluded from the imaging pathways by a 785 nm notch filter. White light (WL) is provided through a commercially available xenon light source (Storz, Germany). The distinctive filter configuration (FIG. 9) allows us to image fluorescence emission with a very high signal to noise (S/N) ratio. The camera simultaneously acquires both white light (WL) and NIR fluorescence images via a GigE interface to a computer. The NIR images are given a pseudo color, and added to the white light image. We tested the camera's ability to detect tumor in nude mice bearing intracranial glioma pre-treated with the commercial CTX-ICG conjugate BLZ-100 (a single ICG conjugated to CTX). The camera, which will be used during intraoperative imaging experiments, is a further optimized version given the acronym SIRIS (Synchronized Near-InfraRed Imaging System). The system was tested using various dilutions of the imaging agent BLZ-100 (Blaze Bioscience) in 5% intralipid solutions. The SIRIS could detect BLZ-100 in picomolar concentrations, and with very high signal to noise ratio (S/N) ratio.

### Example 3—Synthesis and Characterization of Imaging Nanoagents

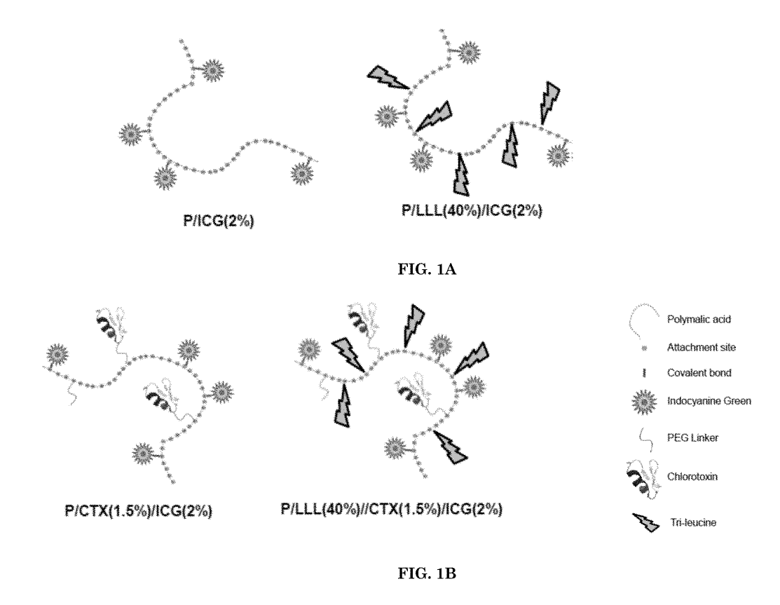

FIGS. 1A-1B are schematic drawings of imaging nanoagents that include polymalic acid (P) conjugated to Iodocyanine Green (IGC) (FIG. 1A) and IGC and Chlorotoxin (CTX) (FIG. 1B). FIG. 1A illustrates the control nanoagent that consists of a polymalic acid (P) with 10 pendent carboxylic groups covalently conjugated with ICG. The structure on the left is a polymalic acid (P) conjugated to IGG (2%), and the structure on the right is a polymalic (P) conjugated to ICG(2%) and tri-leucine (LLL) (40%). FIG. 1B illustrates a tumor specific imaging nanoagent similar to the control molecules shown on FIG. 1A but additionally possessing the tumor specific targeting ligand CTX (1.5%) that is covalently attached via PEG linker (PEG200-PEG5000) to the polymalic acid to ensure high integrity of the nanoagent. The imaging nanoagent shown on FIG. 1B has on average 8 additional molecules of CTX. Tumor specific targeting ligand CTX is covalently attached via PEG linker ensuring high integrity of the nanoagents. The administration of control nanoagent will be used to study the effect of nonspecific tumor accumulation potentially enhanced by EPR effect. Additionally, the control nanoagent will also demonstrate the efficacy of tumor specific targeting ligand.

Imaging nanoagents P/ICG(2%), P/LLL(40%)/ICG(2%) (controls) as shown on FIG. 1A and P/CTX(1.5%)/ICG(2%), P/LLL(40%)/(CTX1.5%)/ICG(2%) (targeted nanoagents) as shown on FIG. 1B were synthesized using polymalic acid, referred to herein as P or PMLA, as a nano-platform. PMLA, has been purified from the culture supernatant of Physarum Polycephalum (>95% purity, Mw 60 kDa, polydispersity P=1.1) (Ljubimova et al., (2014) J Vis Exp; (88), which is incorporated herein by reference as if fully set forth).

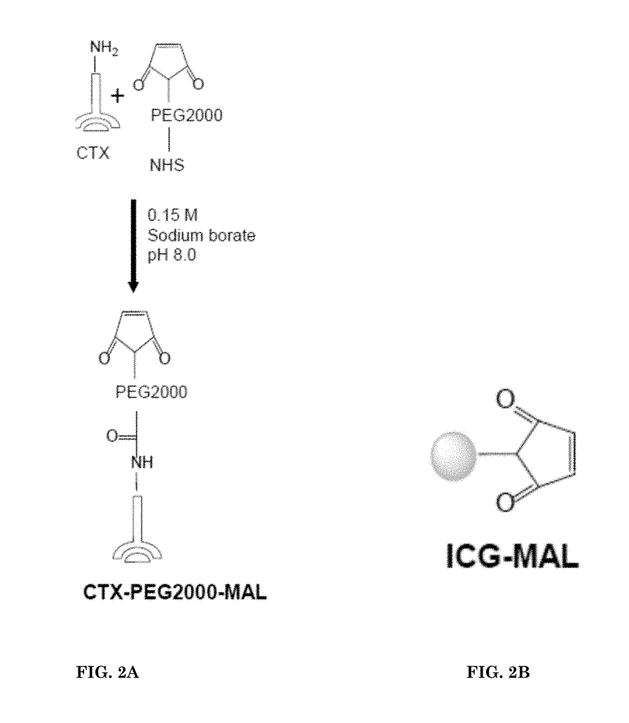

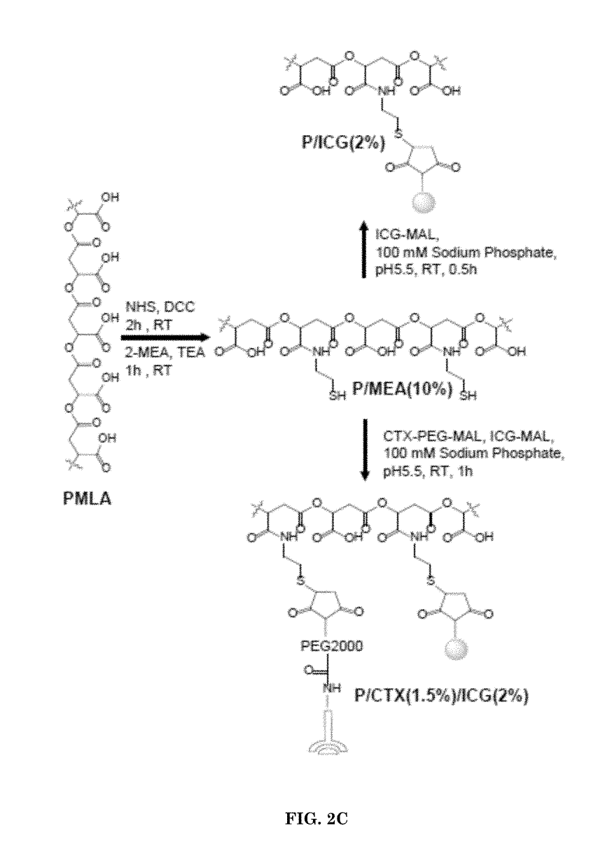

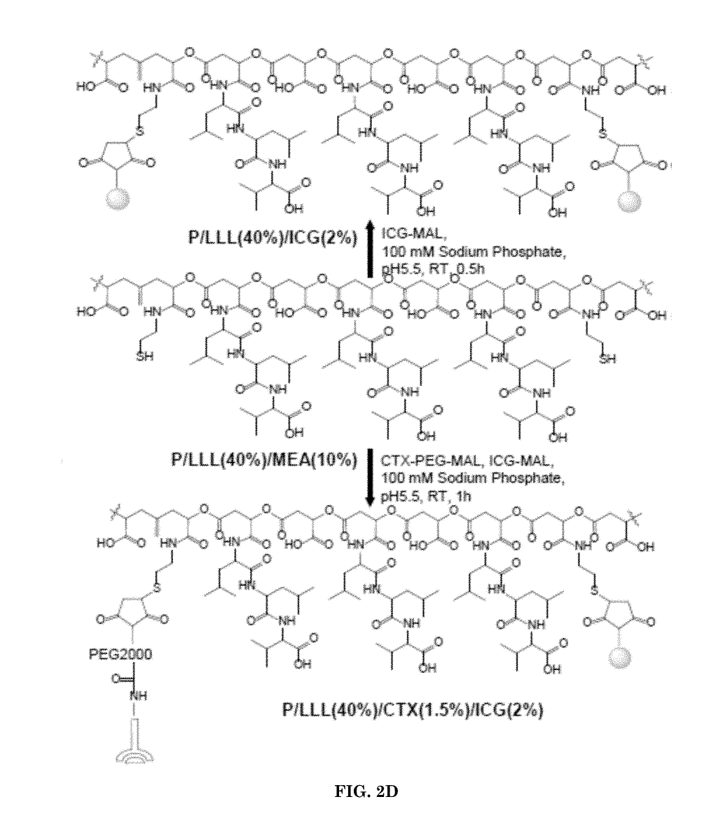

FIGS. 2A-2D illustrate synthesis of nanoagents and intermediates. FIG. 2A illustrates attachment of PEG linker to CTX: CTX was reacted with the bifunctional MAL-PEG2000-SCM (PEG containing maleimide and N-hydroxyl succinamide (NHS), MW 2088 Da in a sodium borate buffer (0.15 M, pH 8.0) for 1 h at room temperature. Product was purified by PD-10 columns. FIG. 2B illustrates commercially available ICG-Maleimide (ICG-MAL). FIGS. 2C-2D illustrate synthesis of PMLA based nanoagents P/ICG(2%) and P/CTX(1.5%)/ICG shown on FIG. 2C, and P/LLL(40%)/ICG(2%) and P/LLL(40%)/CTX(1.5%)/ICG(2%) shown on FIG. 2D. In the first step, Preconjugate was synthesized by conjugating 2-MEA to generate adhere thiol (—SH) groups. In the next step, thiol groups were used to form thioethers with maleimide groups of CTX-PEG2000-MAL and ICG-MAL. Referring to FIG. 2C, the pendant carboxylic groups were chemically activated by the standard NHS/DCC method. Intermediates were prepared by covalently attaching CTX-NH₂ with MAL-PEG2000-SCN linker to form CTX-PEG2000-MAL in borate buffer pH 8.0 and DMF (1:2 mol:mol). Preconjugates were prepared by attaching 2-mercapto-1-ethylamine (MEA) and/or Tri-leucine (LLL) to chemically activated PMLA backbone in DMF/triethyleneamine and following the reaction by thin-layer chromatography and the ninhydrin reaction. Thiol groups of the Preconjugate were used to form a stable covalent bonds (thioethers) with maleimide groups of CTX-PEG2000-MAL and ICG-MAL. The targeted fluorescence agent contains CTX as a targeting moiety as illustrated on FIG. 1B attached at 1.5% of the total malyl residues corresponding to about 7-8 molecules CTX per polymer chain and ICG attached at 2% of total malyl residues corresponding to 10 molecules of ICG. The control agent was synthesized by the reaction of ICG-MAL with Preconjugate and contained 10 molecules of ICG. Excess NHS was hydrolyzed in 100 mM Na phosphate pH 5.5. The remainder of —SH groups on Preconjugate were masked by reaction with pyridyldithiopropionate (PDP). Formation of the products was monitored by sec-HPLC and UV absorbance. It was obtained pure after PD-10 column (60% yield, stored at −20° C.). The synthesized agents are highly water soluble and have the designed composition by chemical group analysis, protein assay and UV quantitative photometry (Ljubimova et al., (2014) J Vis Exp: (88); Ding et al., Int J Mol Sci. 2015; 16:8607-8620, which are incorporated by reference herein as if fully set forth). The hydrodynamic diameter and zeta potential by dynamic light scattering using the Malvern Zetasizer system are shown in Table 1 (SD±10%). All imaging agents were pure when examined by sec-HPLC and dynamic light scattering.

TABLE 1
Summary of imaging nanoagents, their abbreviation and
physicochemical characterization

| | Hydrodynamic | Zeta potential^{b} |
|---|---|---|
| Nanoagents and intermediates | diameter^{a} (nm) | (eV) |
| PMLA | 6.1 (±0.4) | −22.8 (±1.3) |
| Preconjugate | 6.5 (±0.6) | −29 (±0.3) |
| PMLA/ICG (2%)^{c} | 8.5 (±0.8) | −33.1 (±1.2) |
| PMLA/CTX (1.5%)/ICG (2%) | 9.8 (±1.1) | −21.2 (±0.7) |
| PMLA/LLL (40%)/ICG (2%) | 8.2 (±1.4) | −24.8 (±1.2) |
| PMLA/LLL (40%)/CTX (1.5%)/ | 11.82 ((±1.6) | −20.47 (±1.8) |
| ICG (2%) | | |

^{a}Hydrodynamic diameter by number distribution at 25° C. measured in PBS at a concentration of 2 mg/ml, calculated from DLS data by the Malvern Zetasizer software (Malvern Instruments, Malvern, UK), which assumes spherical shapes of particles.
^{b}zeta potential at 25° C. in aqueous solution of 10 mM NaCl at 150 mV.
^{c}composition of nanoconjugates; percentage refers to total number (100%) of pendant carboxyl groups in unsubstituted PMLA.

### Example 4—Spectral Properties of Free and Conjugated ICG

Fluorescence of free ICG was compared with that of conjugated ICG.

Absorbance spectra of ICG in aqueous solution are reported to exhibit concentration dependent shifts of the absorbance spectra (Zhou et al., Bioconjug Chem 2010; 25:1801-1810, which is incorporated herein by reference as if fully set forth).

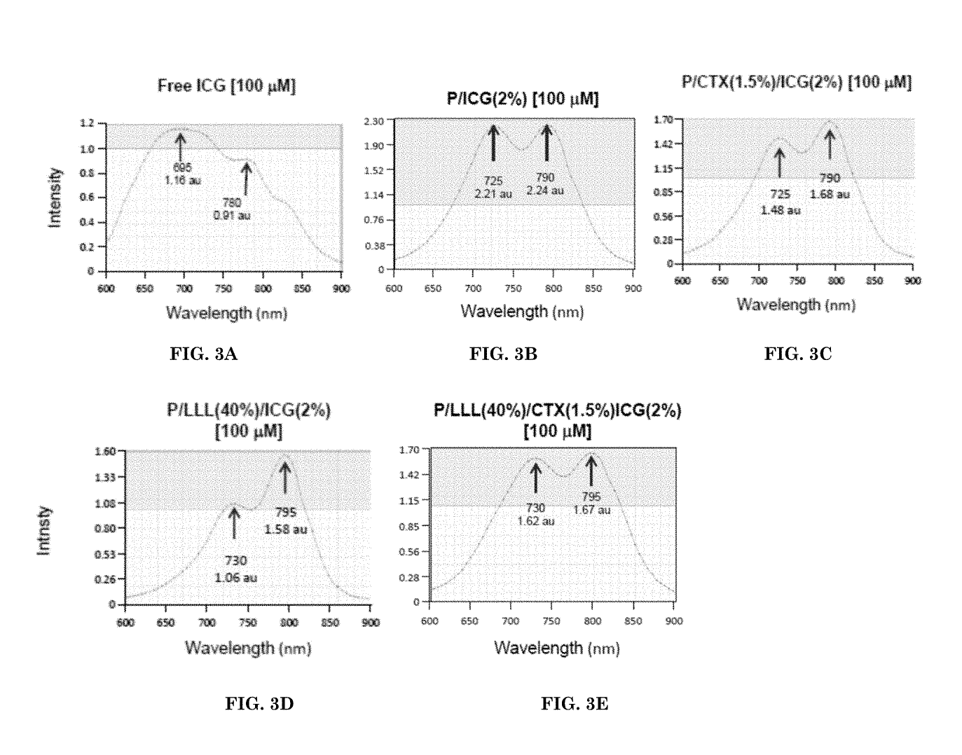

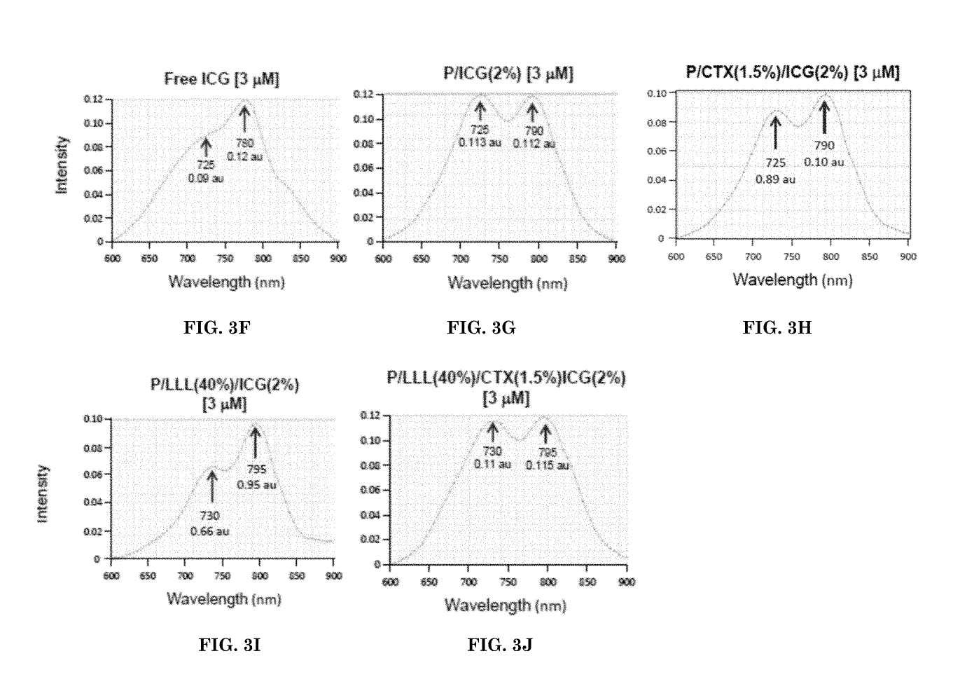

Absorbance of free and conjugated ICG was measured within a wavelength range of 600-900 nm. Stock solution of free ICG was prepared in DMSO at a concentration of 25 mM and sample was diluted in PBS, pH7.4. FIGS. 3A-3J illustrate absorbance spectra of free and conjugated ICG. FIGS. 3A-3E illustrate absorbance at high concentration (100 μM) of free ICG (FIG. 3A), P/ICG(2%) (FIG. 3B), P/CTX(1.5%)/ICG(2%) (FIG. 3C), P/LLL(40%)/ICG(2%) (FIG. 3D), and P/LLL(40%)/CTX(1.5%)/ICG(2%) (FIG. 3E). FIGS. 3F-3J illustrate absorbance at low concentration (3 μM) of free ICG (FIG. 3F), P/ICG(2%) (FIG. 3G), P/CTX(1.5%)/ICG(2%) (FIG. 3H), P/LLL(40%)/ICG(2%) (FIG. 3I), and P/LLL(40%)/CTX(1.5%)/ICG(2%) (FIG. 3J).

Referring to FIGS. 3A and 3F, free ICG shows maximum absorbance at 695 nm at high concentration (100 jM; FIG. 3A) and 780 nm at lower concentration (3 jM; FIG. 3F). Whereas, conjugated ICG to PMLA backbone with or without the presence of CTX did not show difference in the position of absorbance maxima two distinct peaks (725 and 790 nm) were seen at both concentrations as shown on FIGS. 3B, 3C, 3G, and 3H. A 10-nm shift of the peak at the lower wavelength was noticed for conjugated ICG in comparison to the peak at 780 nm for free ICG. Addition of LLL to nanoagents showed increased absorbance intensity of 725 nm peak. A small red shift for the latter peak was noticed for conjugated ICG. Addition of LLL made the red shift moved further by 5 nm as shown on FIGS. 3D, 3E, 3I and 3J. All concentrations are referred as ICG concentrations.

As shown on these figures, at 100 μM ICG the spectrum indicated maximum absorbance at 695 nm wavelength and two other maxima at 780 nm and a third one at higher wavelength, whereas at 3 μM concentration the maximum absorbance was seen 780 nm and a shoulder at 725 nm wavelength. Surprisingly, the spectrum for ICG in the conjugate P/ICG(2%) exhibited two well separated absorbance maxima of similar intensity at 725 nm (shoulder) and 790 nm (maximum) wavelengths at both concentrations 100 μM and 3 jM. The change in the spectrum was accompanied by a more than 2-fold absorbance increase at 100 μM total ICG and by a 30% increase at 725 nm in the case of 3 μM total ICG. The spectra for ICG in the conjugate P/CTX(1.5%)/ICG(2%) were similar exhibiting maxima at 725 nm and 790 nm wavelengths compared with the spectra of P/ICG(2%) except that the absorbance at 725 nm was less (5-10%) than the absorbance at 790 nm wavelength, and the ICG absorbance values were generally lower by 20-40% for P/CTX(1.5%)/ICG(2%) in comparison with P/ICG(2%). Whereas at 100 μM conjugated ICG had higher absorbance than free ICG, they were at 3 μM equal or 20-30% lower than for free ICG. Referring to FIGS. 3D, 3E, 3I and 3J, addition of LLL to nanoagents caused the red shift and two maximums were 730 and 795 nm respectively. Also, the absorbance values were 20-30% higher at 795 peak. Interestingly, addition of CTX to LLL containing conjugates showed almost equal absorbance values at both 730 and 995 peaks.

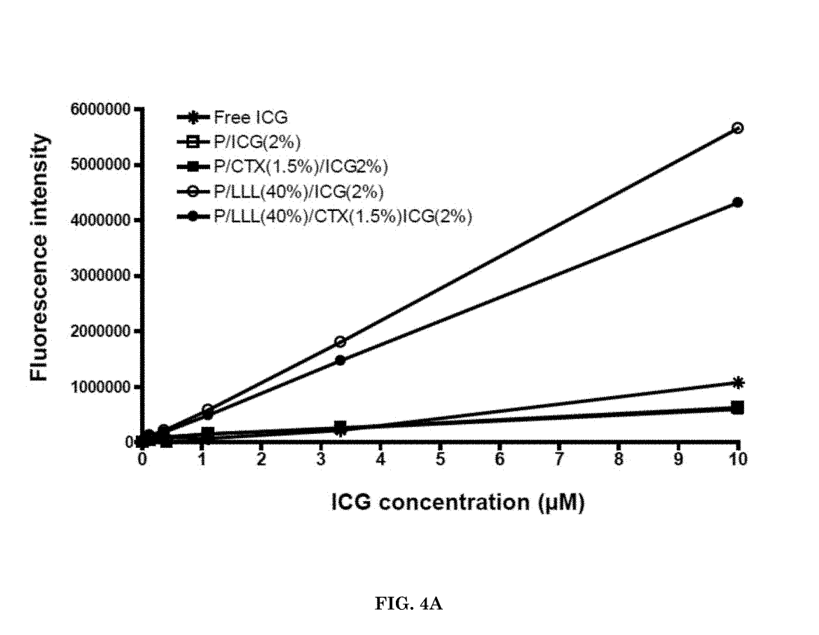

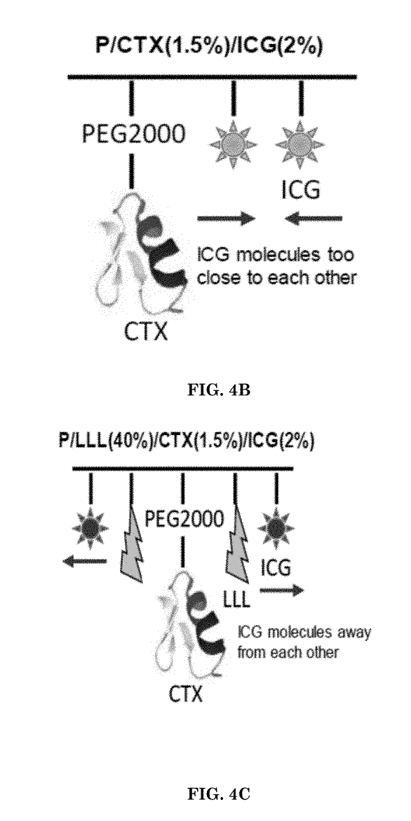

Fluorescence of nanoagent was measured using Odyssey clx at 800 nm channel. FIGS. 4A-4C illustrate fluorescent intensity and properties of nanoagents. FIG. 4A illustrates fluorescence intensity of nanoagents P/ICG(2%) (open square), P/CTX(1.5%)/ICG(2%) (closed square), P/LLL(40%)/ICG(2%) (open circle), P/LLL(40%)/CTX(1.5%)/ICG(2%) (closed circle) and control free ICG (asterisk). The data in FIG. 4A for P/ICG(2%) and P/CTX(1.5%)/ICG(2%) superimpose. FIG. 4B illustrates that weak fluorescence of P/CTX(1.5%)/ICG(2%) may be explained by proximity of ICG molecules to each other. FIG. 4C illustrates that high fluorescence of P/LLL(40%)/ICG(2%) and P/LLL(40%)/CTX(1.5%)/ICG(2%) may be explained by attachment of two ICG molecules not proximal to each other, and separated by tri-leucine LLL. Referring to FIGS. 4A-4C, free ICG was used as a reference. Nanoagents P/ICG(2%) and P/CTX(1.5%)/ICG(2%) showed weak fluorescence compared to free ICG. Nanoagents P/LLL(40%)/ICG(2%) and P/LLL(40%)/CTX(1.5%)/ICG(2%) showed much higher fluorescence compared to free ICG.

Referring to FIG. 4A, when ICG was bound to PMLA, it showed relatively weak fluorescence possibly by self-quenching compared to free ICG. After introduction of hydrophobic LLL to nanoconjugates, no quenching was observed and linear fluorescence increase at all concentrations was noticed. Referring to FIGS. 4B-4C, the quenching effect can be attributed to possible steric factors in the absence of LLL (FIG. 4B), and introduction of hydrophobicity by the leucine side chains as addition of LLL dramatically enhanced the fluorescence (FIG. 4C).

### Example 5—Tumor Visualization by Fluorescence Imaging with Targeted Imaging Nanoagent PMLA/CTX/ICG

The synthesized targeted imaging nanoagents P/CTX(1.5%)/ICG(2%) and P/LLL(40%)/CTX(1.5%)/ICG(2%) along with non-targeted controls P/ICG(2%) P/LLL(40%)/ICG(2%) were tested in vivo using the custom built imaging system. Athymic NCr-nu/nu mice (NCI-Fredrick) were stereotactically injected with 5×10⁴ U87MG human glioblastoma cells in the right basal ganglia. After tumors had grown to an appropriate size (2-4 mm), mice received a tail vein injection of either control or targeted agent at a dose of 200 nmol/kg (in terms of ICG concentration). No behavioral or physical abnormalities were observed. Images were acquired at 2, 4, 8, 12 24 and 48 hours. Organs were isolated after and visualized using the custom-built imaging system. Tumors were visualized by custom built SIRIS (Synchronized near-InfraRed Imaging System) under white light mode. White light mode is comprised of a light engine with 4 LEDs (red, blue, green and cyan). The details of the imaging system used for real time tumor visualization and imaging is described in Kittle, D. S., Mamelak, A., Parrish-Novak, P. E., Hansen, S., Patil, R., Gangalum, P. R., Ljubimova, J., Black, K. L. and Butte, P. “Fluorescence-Guided Tumor Visualization Using the Tumor Paint BLZ-100”, Cureus 6:e210 (2014).

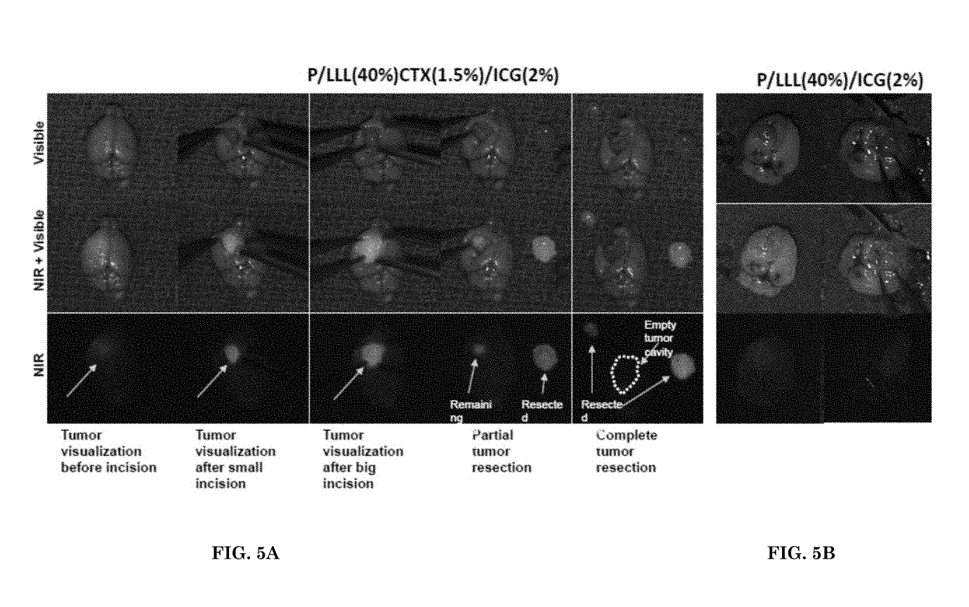

FIGS. 5A-5B are photographs of tumor visualized by targeted nanoagent P/LLL(40%)/CTX(1.5%)/ICG(2%) (FIG. 5A) and control nanoagent P/LLL(40%)/ICG(2% (FIG. 5B)). The images at the top of the panels marked “Visible” were recorded under visible (white light) of SIRIS, the images in the middle of the panels marked “Visible+NIR” show the same areas of brain assumed on the basis of white light and clearly visible under near-infra-red (NIR) mode and were superimposed on the monitor.

The images at the bottom of the panels marked “NIR” show the same areas of brain as shown at the top and middle of the panels but were recorded under fluorescence (NIR) only. FIG. 5A illustrates tumors visualized before incision (left panel, on the left), after small incision (left panel, on the right), after big incision (middle panel, on the left), after partial tumor resection (middle panel, on the right), and after complete tumor resection (right panel) As shown on FIG. 5A, targeted nanoagent P/LLL(40%)/ICG(2%) was selectively accumulated only in the tumor area as evident by clear demarcation (arrows) with no fluorescence detectable in the normal brain. Area highlighted by strong fluorescence was histologically evaluated by H&E staining to confirm the malignancy. FIG. 5B illustrates that the control nanoagent P/LLL(40%)/ICG(2%) failed to accumulate in tumor as very weak fluorescence was observed. Referring to FIG. 5A, tumors could not be not visualized by white light before incision, although the approximate area of tumor formation was expected based on the tumor inoculation site. Under NIR mode, the tumor could be localized by virtue of P/LLL(40%)/CTX(1.5%)/ICG(2%) fluorescence representing tumor drug accumulation before excision. In accordance with tumor targeted delivery by CTX, the high fluorescence intensity was detected specifically at the site of the tumor for targeted nanoagentss but not in the surrounding healthy parts of the brain. Thus, the tumor delineation was sharp and clear (marked by arrows). Referring to FIG. 5B, very little or no fluorescence was seen with the control nanoagent P/LLL(40%)/ICG(2%) in tumor area. All other vital organs showed minimum to no fluorescence. These results demonstrated the ability of nanoagents to selectively target preclinical glioblastoma and the suitability for use in precise and selective microsurgical resection.

### Example 6—Pharmacokinetics (PK)

For assessment of serum half-life, the fluorescence of ICG which covalently bound to nanoagent was utilized. Blood was collected from mice at 0.083, 0.5, 1, 2 3, 6, 8, 12, 16, 24, and 48 hours after injection of 150 μl of nanoagent at a dose of 200 nmol/Kg (ICG dose). Blood was centrifuged and the serum was collected for analysis. The blood serum was mixed with 1:1 ratio of PBS, added to a 96 well clear bottom plate and scanned using the Odyssey Clx scanner at 800 nm channel. The ICG could be readily detected with almost no background and quantified in small volume blood samples. Exponential decay analysis of targeted nanoagent revealed an elimination half-life of 1.2 hours.

### Example 7—Biodistribution of Imaging Nanoagents

An assay, in which a NIR fluorescence scanner Odyssey Clx was used, was utilized to quantitatively assess ICG signal at 169 micrometer microscopic resolution.

Tumor bearing mice were injected through the tail vein with 150 μl of nanoagent at a dose of 200 nmol/Kg (ICG dose). Mice were sacrificed at 2, 4, 8, 12, 24 and 48 h after dosing and whole organs including brain, lung, kidney, liver, heart and spleen were removed. Brain tumors were carefully removed under real time fluorescence guided resection as shown on FIG. 5A along with a similar size normal brain from other hemisphere as a reference. A portion of tissue from lung, kidney, liver, heart and spleen was carefully cut from the whole organ. All the samples were weighed and transferred in a 2.0 ml Eppendorf tubes. PBS was added to the organ containing tubes at a 1:10 weight: volume ratio and samples were ultrasonicated to form a homogeneous solution. Samples were transferred to a 96 well clear bottom plate and scanned using the Odyssey Clx scanner at 800 nm channel. At least triplicates were used for data recording. A standard curve was prepared separately for each organ using known concentrations of nanoagent and was used for quantification of nanoagent in each organ. For standard curve preparation, tissues from animals injected with PBS were used. Signal strength was very good as signal to noise ratio was very high with background signal being less than 0.01%.

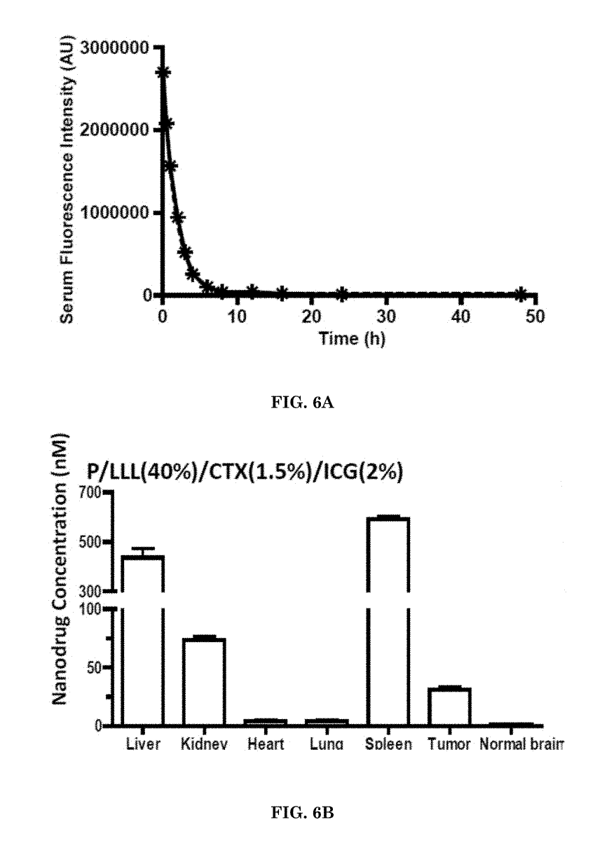

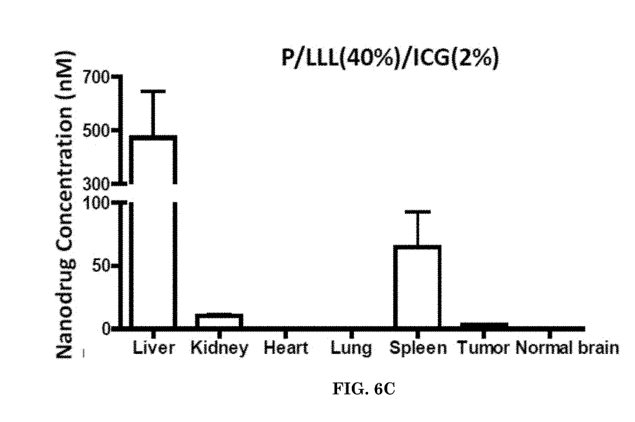

FIGS. 6A-6C illustrate pharmacokinetics measured as fluorescence intensity of the targeted imaging agents in serum and localization of the targeted and non-targeted imaging nanoagents. FIG. 6A illustrates fluorescence intensity for targeted nanoagent in serum. FIG. 6B illustrates concentration of the nanoagent P/LLL(40%)/CTX(1.5%)/ICG(2%) in liver, kidney, heart, lung, spleen, tumor and normal brain. FIG. 6C illustrates concentration of the control nanoagent P/LLL(40%)/ICG(2%) in the same organs as shown in FIG. 6B. The figures illustrate pharmacokinetics and indicate serum half-life. Serum PK of the targeted nanoagent was followed using ICG fluorescence and PK half-life was found to be 1.2 h. FIG. 6B illustrates localization of the targeted nanoagent in organs. Highest amounts of the drug were observed in spleen and liver followed by kidneys and tumor. Heart and lung showed very low amount and normal brain showed the lowest drug concentration. Referring to FIG. 6C, the control nanoagent P/LLL(40%)/ICG(2%) showed very weak accumulation in tumor area.

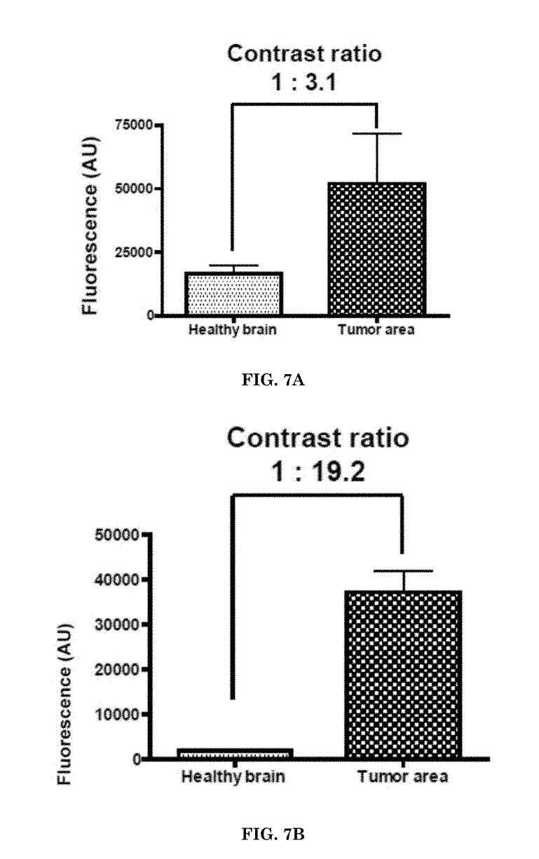

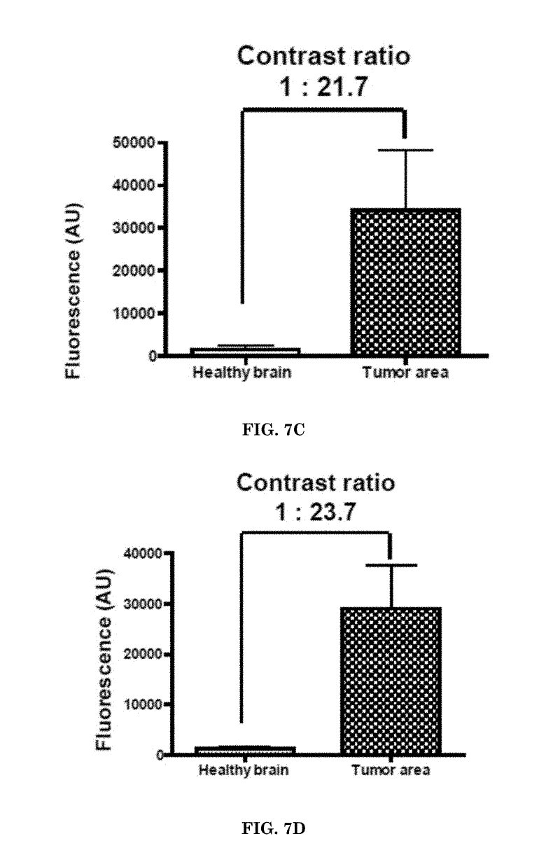

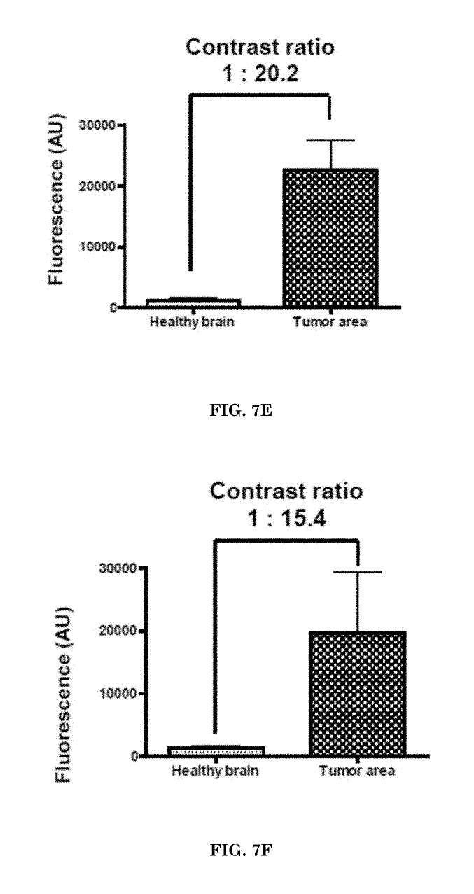

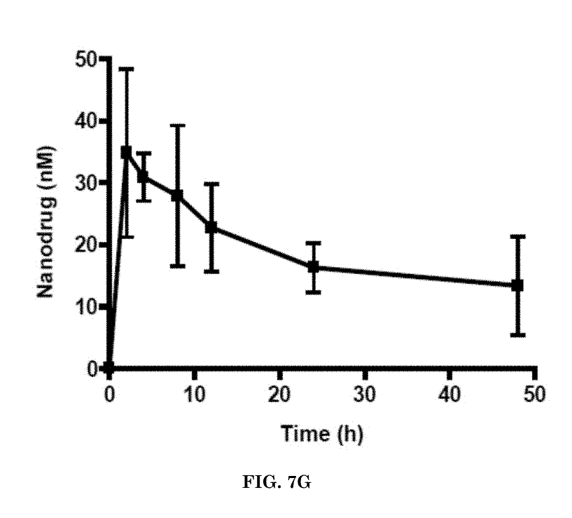

FIGS. 7A-7G illustrate accumulation of the imaging nanoagent after administration to a subject. FIGS. 7A-7F illustrate accumulation of the imaging nanoagent as function of time after administration. FIG. 7A illustrates accumulation of the imaging nanoagent P/LLL(40%)/CTX(1.5%)/ICG(2(%) and contrast ratio in brain tumor vs. surrounding healthy brain at 2 hours. FIG. 7B illustrates accumulation of the imaging nanoagent and contrast ratio at 4 hours. FIG. 7C illustrates accumulation of the imaging nanoagent and contrast ratio at 8 hours. FIG. 7D illustrates accumulation of the imaging nanoagent and contrast ratio at 12 hours. FIG. 7E illustrates accumulation of the imaging nanoagent and contrast ratio at 24 hours. FIG. 7F illustrates accumulation of the imaging nanoagent and contrast ratio at 48 hours. FIG. 7G illustrates accumulation of the imaging nanoagent in the tumor as function of time. Nanoagent was administered via I.V. tail vein injections. A ratio of 1:3.1 was seen in brain tumor vs surrounding healthy brain at 2 h whereas contrast ratio was much higher at 4-48 hours. FIG. 7G illustrates that significant nanoagent was seen in tumor at from 2-48 hours, with highest drug concentration of 34.84 (±7.78) nM at 2 h as shown in FIG. 7A and highest contrast ratio of 1:23.7 at 12 hours as shown on FIG. 7D. Significant contrast ratio was maintained up to 48 hours.

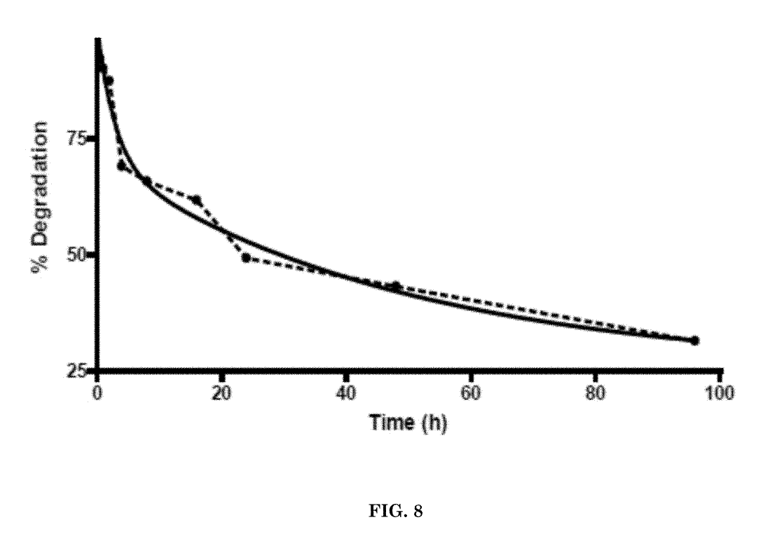

FIG. 8 illustrates stability of the targeted nanoagent in tumor. Nanoagent showed good tumor stability with half-life of 4 hours. In contrast, nanoagent degradation in serum monitored by High Performance Liquid Chromatography (HPLC) had a life time of 10 h.

The imaging system that was used for real time tumor visualization and imaging is described in Kittle, D. S., Mamelak, A., Parrish-Novak, P. E., Hansen, S., Patil, R., Gangalum, P. R., Ljubimova, J., Black, K. L. and Butte, P. “Fluorescence-Guided Tumor Visualization Using the Tumor Paint BLZ-100”, Cureus 6:e210 (2014), incorporated herein by reference as if fully set forth. For example, the equipment suitable for NIR imaging may be the Synchronized near-InfraRed Imaging System (SIRIS) that includes NIR Laser (785 nm), Laser Clean-up filter, Notch Beam Splitter, the source of white light, camera line, Basler Camera (11.26×5.98 mm sensor, 340 fps), Edmond #67-716 (35 mm focal length VIS-NIR lens), Notch filter (785 nm), two fold mirrors, collimating lens, diffuser, and windows for excitation and imaging. This unit measures 7.75″×3.74″×2.06″ and weigh around 3.8 lbs and can be attached to commercial endoscope holders. With the focal distance of 45 cm it sits well outside the surgical field and allows instruments and specimen to be easily passed under it during the surgical resection. The camera output is connected to an image processing computer and then fed to high definition video monitor for display.

FIG. 9 illustrates imaging systems filter configurations. The use of very narrow band laser light to excite ICG at the wavelength of 785 nm aided by use of a cleanup filter to allow for maximum excitation efficiency. In conjunction, a notch filter in front of the camera is able to remove the excitation light from the image thus capturing only the fluorescence emission for the target. This configuration allows imaging system to image fluorescence with maximum efficiency with high signal-to-noise ratio.

### Example 8—NIA Permeation of Blood Brain (Tumor) Barrier (BBB) and Subcellular Distribution in GBM

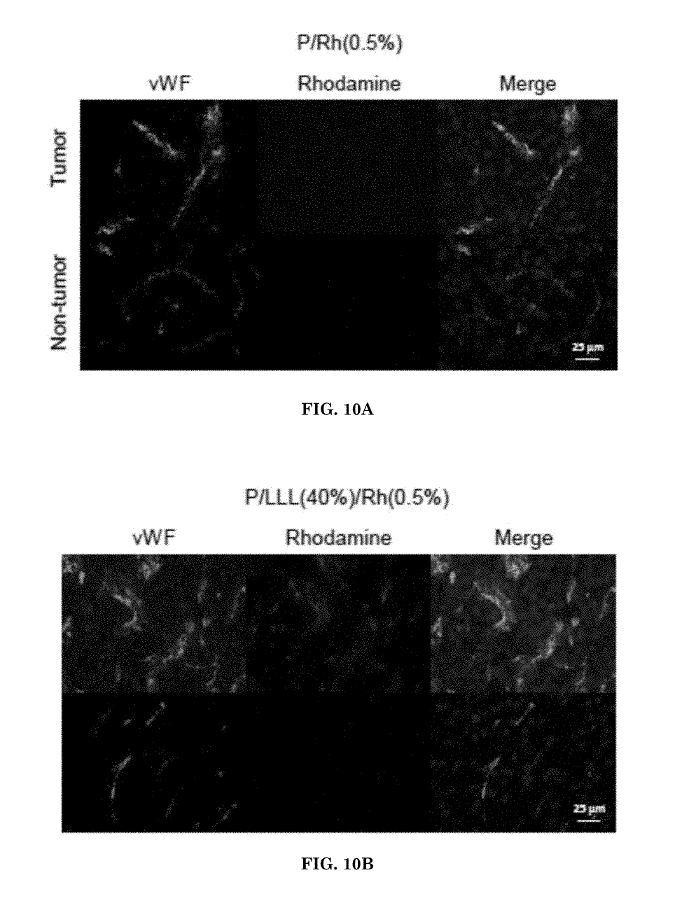

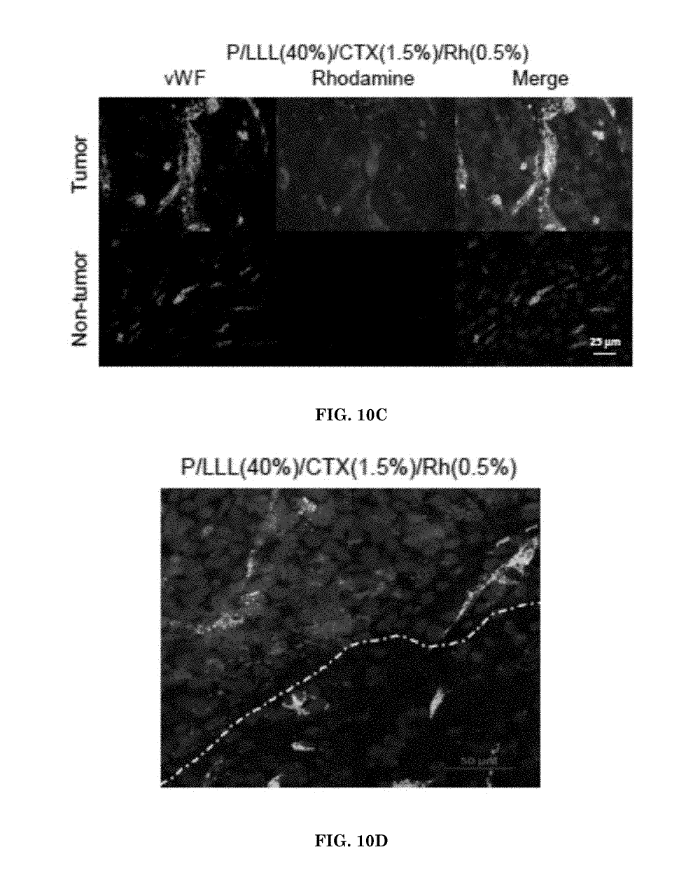

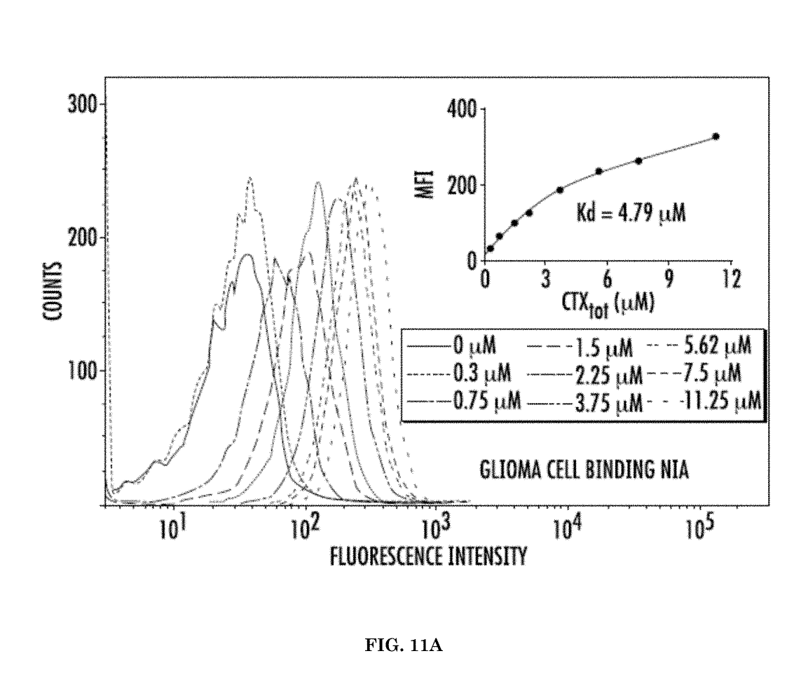

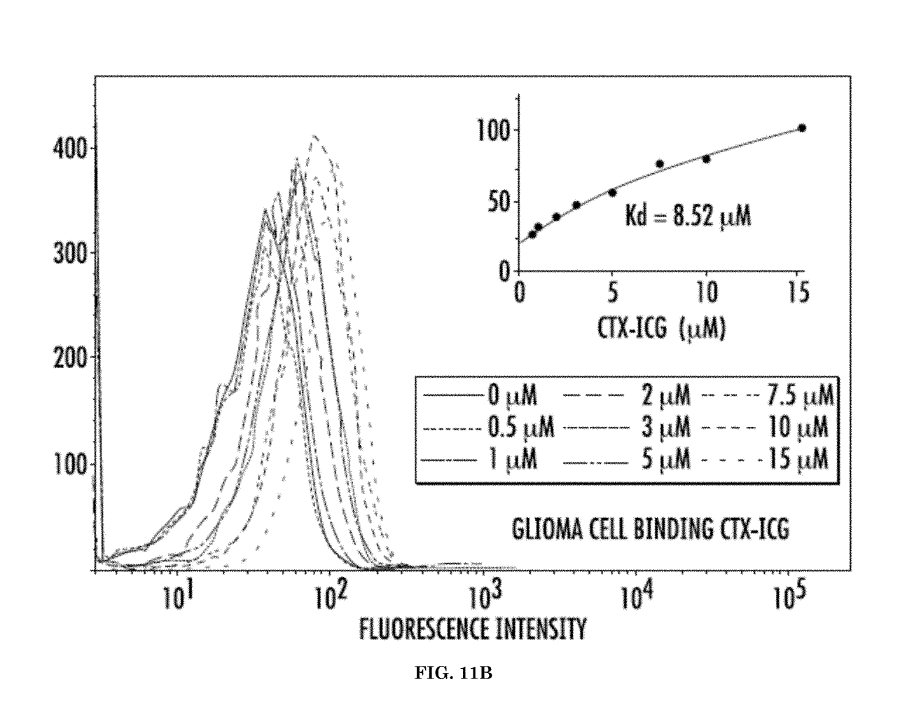

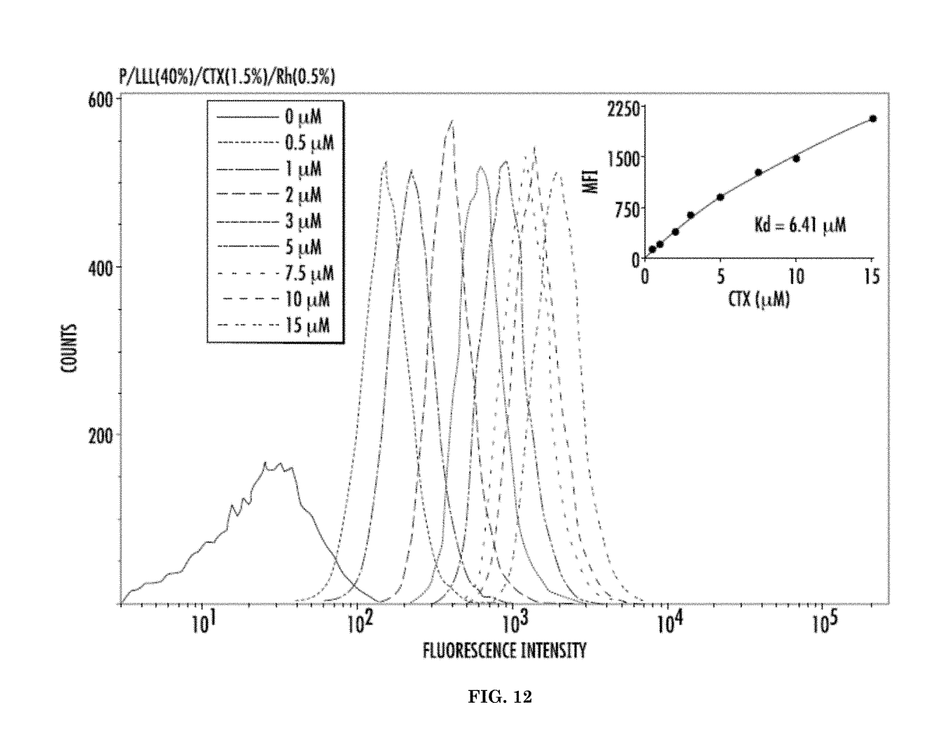

To prove internalization, the cellular distribution of the imaging agent in xenogeneic GBM and especially in the tumor cells was studied by ex vivo fluorescence microscopy of glioblastoma and healthy brain sections using imaging nanoagent (NIA(Rh)) which had (2%) ICG substituted by rhodamine (0.5%). According to evidence for similar binding of NIA(Rh) and NIA by flow cytometry as shown on FIG. 12. The substitution improved the staining intensity and contrast in conventionally equipped fluorescence microscopes. The staining by NIA(Rh) was compared for tumor-free brain and tumor sections. FIGS. 10A-10D illustrate Ex vivo fluorescence microscopy showing extravasation of NIAs across BBB into tumor cells and not into healthy brain regions.

FIGS. 10A-10D illustrate tumor and brain sections 16 hours after iv injection of nanoagent containing rhodamine (Rh) into mouse tails of animals. FIG. 10A illustrates P/Rh(0.5%). FIG. 10B illustrates P/LLL(40%)/Rh(0.5%). FIG. 10C illustrates P/LLL(40%)/CTX(1.5%)/Rh(0.5%). FIG. 10D shows intense distribution of lead nanodrug P/LLL(40%)/CTX(1.5%)/Rh(0.5%) stained tumor cells and vessels along tumor margin. White dotted line represents tumor margin 16 h after iv injection into mouse tails. In the imaging agent P/LLL(40%)/CTX(1.5%)/Rh(0.5%), ICG had been replaced by rhodamine (red color) and the resulting imaging agent had a similar dissociation constant Kd for glioma cell binding as did the original imaging nanoagent. Vessels are stained for von Willebrand factor (WF; green) and nuclei by DAPI (blue). Merge mode (yellow) shows superposition of capillary and imaging nanoagent staining. Red color distribution indicates fluorescence in vesicular and was also diffusely distributed. The degrees of fluorescence were still maintained in the vascular at 16 h after injection of P/LLL(40%)/Ph(0.5%), although PK t1/2=75 min. for NIA. Intense distribution of the imaging nanoagent NIA(Rh) resides specifically in the tumor area along tumor margin indicated as a white dotted line shown on FIG. 10D.

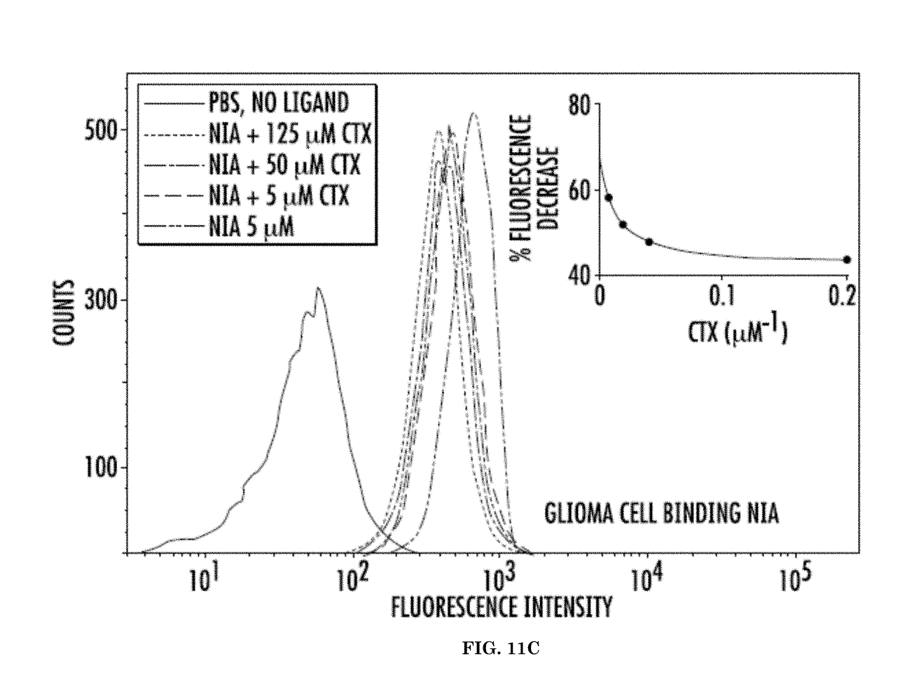

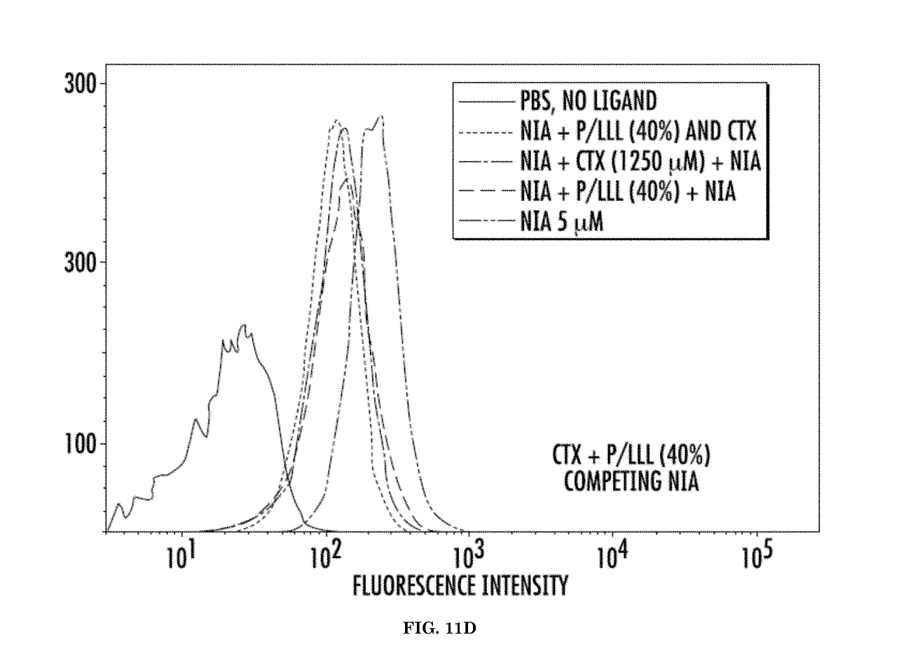

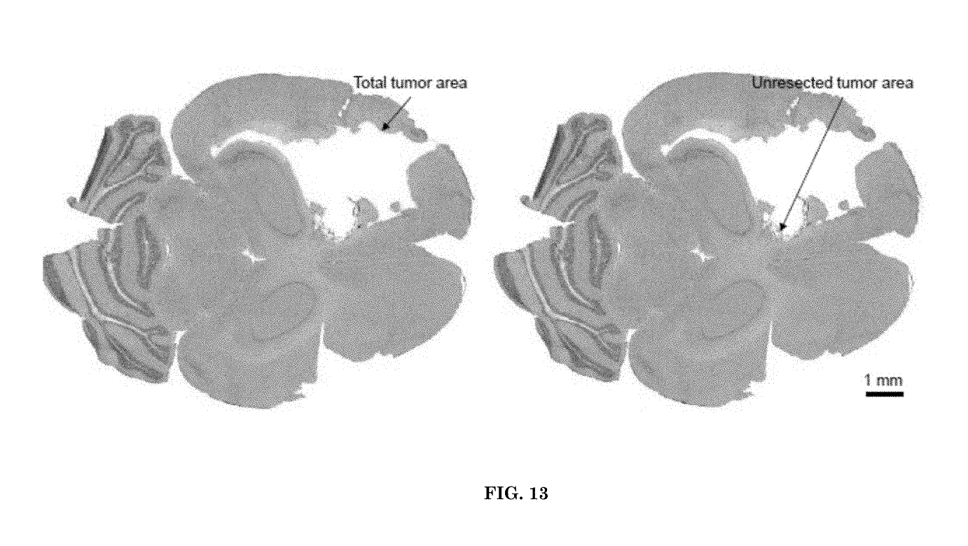

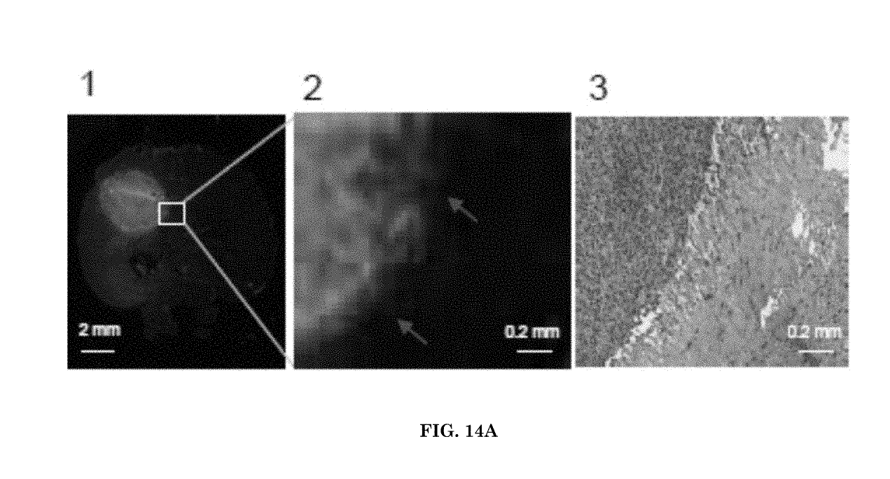

Referring to FIG. 10C, the construct P/LLL(40%)/CTX(1.5%)/Rh(0.5%), or NIA(Rh), exhibited fluorescence inside and outside vascular in tumor cells. In the absence of CTX, P/LLL(40%)/Rh(2%) showed some fluorescence which merged with the anti-vWF-stained vessels as shown on FIG. 10B. Sections probed with P/Rh(0.5%) are devoid of fluorescence staining (FIG. 13). As shown on FIG. 10A, tumor-free brain remained unstained in all cases. The staining by NIA(Rh) that did not contain CTX and persisted long after clearance from the blood stream at 16 h after iv injection, was hypothesized to reflect interactions with P/(LLL40%) binding sites on the NIA molecule found also for NIA(ICG) by flow cytometry. Because of their restricted location to vascular, the finding could indicate that the sites were not active in internalization if CTX was not part of NIA. In contrast, tumor cells, in particular in glioma margins contained a large number of fluorescent vesicles and diffuse fluorescence when injected with NIA(Rh) as was shown on FIG. 10D. The results indicate that the rhodamine substituted NIA permeated the tumor BBB in a CTX dependent fashion as diffuse and/or particle-like deposits in the cells. Supported by the similarity in Kd values for NIA(Rh) and NIA(CTX) (flow cytometry FIGS. 11A-11D and FIG. 12, the results obtained by fluorescence microscopy are consistent with a glioma specific imaging that involves multiple cell binding sites and internalization pathways. Several different binding sites for NIA and mixed mechanisms of uptake into cells could explain the intense staining and the long residence time specifically to the tumor. Permeation through tumor BBB was not investigated here, but it is evidenced by the CTX selectivity referenced for staining GBM.

### Example 9—Interaction of Imaging Nanoagent P/LLL(40%)/CTX(1.5%)/ICG(2%) with Glioma Cells

The degree of glioblastoma imaging by fluorescence depends on the binding of the imaging nanoagent (NIA) P/LLL(40%)/CTX(1.5%)/ICG(2%) to the surface of glioma cells. Several kinds of interactions could be possible based on the diverse multiple groups, CTX, LLL, and ICG, and could empower selective binding to several tumor surface molecules and invoke more than one internalization pathways into glioma cells. To shed light on modes of interactions, the studies were focused on binding to the cell surface of glioblastoma cells using flow cytometry, focusing on contributions of P/LLL(40%), CTX and ICG.

### Ligand Binding to Glioma Cell Surface.

Printouts for the binding of P/LLL(40%)/CTX(1.5%)/ICG(2%) and CTX/ICG are shown as histograms in FIGS. 11A-11B.

FIGS. 11A-11D illustrate binding of the imaging nanoagent (NIA) P/LLL(40%)/CTX(1.5%)/ICG(2%) and CTX/ICG to U87 MG glioma cells indicated by mean fluorescence intensity (MFI) of ICG measured by flow cytometry. FIG. 11A illustrates flow cytometry histogram for binding of NIA as a function of concentration of total CTX, CTXtot. The following concentrations were tested: 0 μM (solid line), 0.3 μM (dashed line), 0.75 μM (dotdashed line), 1.5 μM (longdashed line), 2.25 μM (dotted line), 3.75 μM (dash-twodotted line), 5.62 μM (twodashed line), 7.5 μM (longdashed line) and 11.25 μM (raredashed line). Inset of this figure shows median fluorescence intensity (MFI) as a function of total concentration of CTX (=CTXtot). Kd=4.79 μM, operational dissociation constant is calculated for CTXtot. Assuming NIA contains on average 7.75 molecules CTX, the dissociation constant for the cell NIA complex is calculated for each attached CTX as Kd(NIA)=Kd(CTXtot)/7.75=0.618 μM. FIG. 11B illustrates flow cytometry histogram for binding of CTX-ICG as function of total concentration of CTX. The following concentrations were tested: 0 μM (solid line), 0.5 μM (dashed line), 1 μM (dotdashed line), 2 μM (longdashed line), 3 μM (dotted line), 5 μM (dash-twodotted line), 7.5 μM (twodashed line), 10 μM (longdashed line) and 15 μM (raredashed line). Inset of FIG. 11B shows median fluorescence intensity (MFI) as a function of concentration of CTXtot. Kd, the operational dissociation constant calculated for the dissociation of the cell CTX-ICG complex. FIG. 11C illustrates flow cytometry histogram for CTX (not fluorescent) competing with binding of NIA (content 5 μM CTXtot) at various concentrations of competing CTX. The following combinations were tested: PBS no ligand (solid line), NIA+125 μM CTX (dashed line), NIA+50 μM CTX (dotdashed line), NIA+5 μM (longdashed line), and NIA 5 μM (dash-twodotted line). Inset of FIG. 11C shows the decrease in median fluorescence intensity as function of inverse concentration, CTX⁻¹. The abscissa intercept indicates the maximum level of fluorescence decrease due to competition at extrapolated infinite concentrations of CTX. The same method was used to extrapolate for infinite concentrations in the competition of NIA by P/LLL(40%). FIG. 11D illustrates flow cytometry histogram for the mixture of CTX (125 μM) and P/LLL(40%) (12.5 μM), both not fluorescence labelled, competing with binding of NIA (content 5 μM CTXtot). The following combinations were tested: PBS, no ligand (solid line), NIA+P/LLL(40%) and CTX (dashed line), NIA+CTX(1250 μM)+NIA (dotdashed line), NIA+P/LLL(40%)+NIA (longdashed line), and NIA 5 μM (dash-twodotted line).

The saturation curves in the insets were calculated as average fluorescence intensities as function of overall CTX, (CTXtot), concentrations. Curves in insets of FIGS. 11A-11B were computed for single site binding modes with best fits for apparent dissociation constant Kd (NIA, CTXtot)=4.79 μM (FIG. 11A, NIA, when varied total concentration of CTX, CTXtot), and Kd (CTX-ICG, CTXtot)=8.5 μM (FIG. 11B). Each molecule of NIA contains on average 7.75 groups of CTX. Assuming that each residue of CTX contributes incremental binding, a global dissociation constant for NIA is assigned Kd (NIA)=Kd (NIA, CTXtot)/7.75=0.62 μM (FIG. 11A) and K_{d} (CTX)=Kd (CTX-ICG, CTXtot) 8.5 μM. According to its chemical synthesis, NIA has the formal composition P/LLL(40%)/CTX(1.5%)/ICG(2%). The functional groups, P/LLL(40%), CTX, ICG can be considered as each contributing to the binding of NIA, and in the case of separate molecules, groups P/LLL(40%) and CTX can be tested alone and in a mixture for their potential to inhibit the binding of glioma cells. Since the inhibitors are not fluorescence labeled, the inhibition would be indicated by the reduction of MFI if NIA is the competed ligand. A decrease in mean fluorescence intensity was indeed observed (printout histogram FIGS. 11C-11D). The result of competing 5 μM NIA (100% fluorescence intensity) with 125 μM P/LLL(40%) was 41-46% decrease, with 125 μM CTX 54-56% decrease, and with combined 125 μM P/LLL(40%) plus 125 μM CTX the decrease was 56-58%. The decrease obtained by extrapolation of (concentration)⁻¹ to zero (simulating the effect of infinitely high concentration of competitor, example for CTX in FIG. 11B), was not substantially higher. In addition, the removal of the solutionin in the competition reaction containing the mixture of NIA plus competitors, and substitution with PBS at the end of the competition experiment, did not diminish the remaining % of fluorescence intensity and indicated the absence of uncontrolled fluorescence. The results indicated that NIA could not be fully displaced from binding to glioma cells. The incomplete degree of displacement by single and double competition is best explained by mixed competitive/noncompetitive inhibition. The competitors bind to sites recognizing P/LLL(40%), CTX. A further site(s) bind ICG, indicated by 50-60% residual fluorescence intensity for CTX competition with CTX-ICG and by exposure of cells to ICG. The sites are independent, allosterically coupled or partially overlap NIA bindings sites. Whatever the explanation, the results indicate that NIA's specific binding to glioma cells involved three or more sub-sites.

FIG. 12 illustrates binding of NIA P/LLL(40%)/CTX(1.5%)/Rh(0.5%) to glioma cells measured via mean fluorescence intensity (MFI) of Rh by flow cytometry. The histogram printout is evaluated as mean fluorescence intensity as function of total CTX concentration (CTXtot) in the figure inset. The following concentrations were tested: 0 μM (solid line), 0.5 μM (dashed line), 1 μM (dotdashed line), 2 μM (longdashed line), 3 μM (dotted line), 5 μM (dash-twodotted line), 7.5 μM (raredashed line), 10 μM (long-and-raredashed line) and 15 μM (twodashed line). Non-linear curve fitting on a single site binding model indicates operational Kd 6.41 μM, which compares to operational Kd=4.79 μM of the ICG containing NIA, P/LLL(40%)/CTX(1.5%)/ICG(2%).

### Example 10—Fluorescence Staining of Glioblastoma in Nude Mice

The performance of the imaging nanoagent, P/LLL(40%)/CTX(1.5%)/ICG(2%), was tested in vivo using the SIRIS imaging camera. The test followed iv injection into the tail of nude mouse and surgery under the camera at optimal settings of camera and 4 h after injection. Tumor was located under NIR by its pseudo color (blue fluorescence). After incision and removal of tissue under NIR light, the tumor appeared with sharp boundaries and could be resected in a few steps. Very little or no fluorescence was seen with the control P/ICG(2%). After resection, other vital organs showed maximum to no fluorescence depending on involvement in blood clearing. Intact and resected brain was isolated for inspection of resection quality.

### Example 11—Tumor Resection and Resection Precision

A cohort of mice carrying preclinical GBM at comparable tumor size, were injected with the imaging nanoagent (NIA) P/LLL(40%)/CTX(1.5%)/ICG(2%), or PBS (control group), euthanized and brains harvested. For optimization of resection methods, tumors were resected from isolated brains in absence and presence of the NIA fluorescence 4 h after injection. After resection, brains were fixed, sectioned in three different areas (top middle and deep) and stained with H&E for tumor tissue identification under the microscope. For each section, 2-dimensional regions of interest (ROI) were measured (FIG. 13) and related to the area for tumor in resected samples using the equation % Resection=100×(Total tumor area−Sum of ROI)/Total tumor area). FIG. 13 illustrates resection of tumor and evaluation of precision by microscopic inspection of H & E stained sections. An ex vivo H & E stained section is shown for measurement of the area. A region of interest (ROI) was drawn around tumor perimeter to determine total tumor volume. Similarly, ROI was drawn around leftover tumor area to determine remaining tumor. % resection is calculated in top, middle and deep tumor sections.

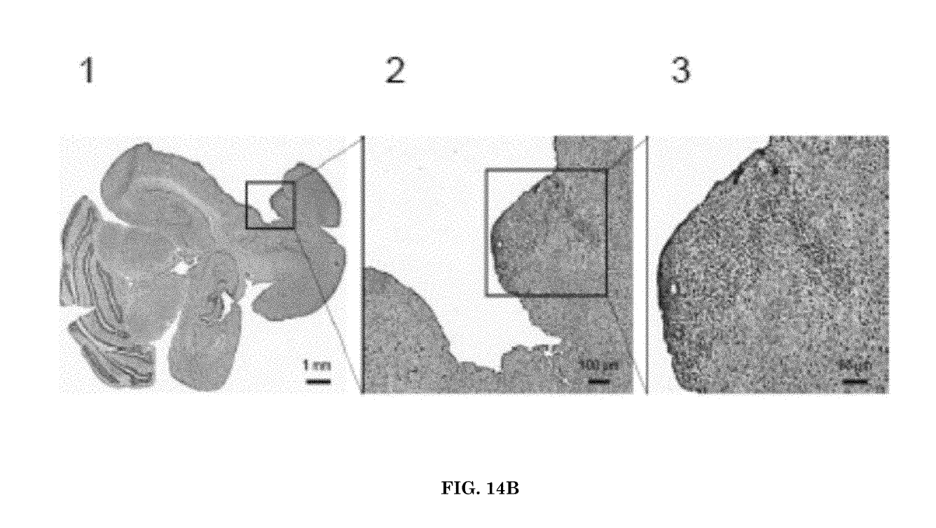

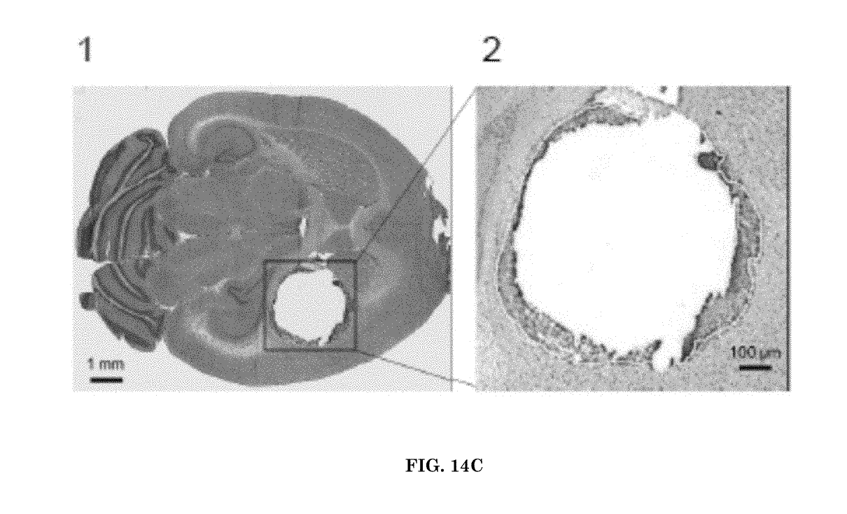

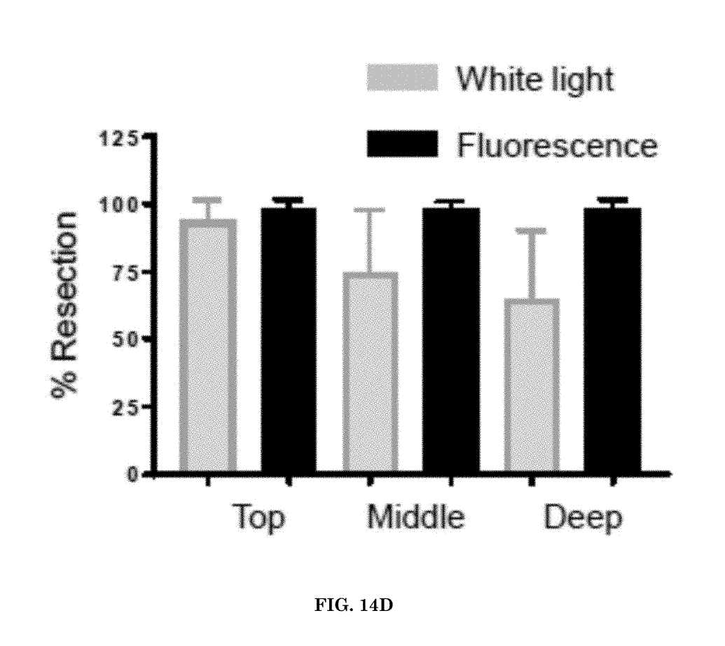

FIGS. 14A-14D illustrate U87 MG GBM xenografts after NIA-guided resection. Precision of tumor resection and interference with tumor infiltration. FIG. 14A, panel 1, illustrates, tumor slice (8 micron deep) of NIA, P/LLL(40%)/CTX(1.5%)/ICG(2%), 4 h after i.v. injection of the NIA, visualized under Odyssey ELX; panel 2 illustrates magnification of tumor border to brain exhibiting interdigitation (arrows) into tumor-free tissue; panel 3, illustrates tumor H&E staining in border regions exhibiting tumor interdigitation into brain for comparison with panels 1 and 2. FIG. 14B, panels 1, 2, and 3, illustrates examples of the tumor fragment (infiltrating tumor cells) remaining after resection under NIR fluorescence of a NIA injected mouse. FIG. 14C, panels 1 and 2, illustrates brain resection for a PBS injected mouse under white light for estimation of resection precision. FIG. 14D illustrates efficiency of tumor resection under white light and NIR. H & E analysis was performed after section brain tissue in top, middle and deep areas. Quantification was performed after analyzing H& E sections to determine tumor volume. Referring to FIG. 14D, the degree of resection under NIA fluorescence light was 98.4±3.1% in top sections declining only marginally to 98.1±3.3% in lower section. In contrast, under white light resection was 93.9±7.0% in top sections and declined to 64.7±23.3% in lower sections. Under NIR light, the non-resected tumor portions were of small size and contained interdigitating parts of the tumor as shown on FIG. 14B, panels 1, 2 and 3. The low precision under white light in middle and low sections followed increased standard deviations. Under white light, large portions of tumor of increased sizes remained not resected in the lower sections, or tumor cell layers along the border that would have been recognized under the fluorescent light. The variability in precision increased from top to low sections and mirrors the increased difficulty to control resection in remote positions as shown on FIG. 14D. Under NIR light, tumor fragments were of small size and attached to tumor free brain by cell infiltration (example shown in FIG. 14B, panels 1, 2 and 3). It is possible that enhanced attachment and less visibility due to reduced number of fluorescent tumor cells bellow detection limit of the SIRIS camera system provoked the escape from resection in these regions.

### REFERENCES

1. Noone A M, Howlader N, Krapcho M, Miller D, Brest A, Yu M, Ruhl J, Tatalovich Z, Mariotto A, Lewis D R, Chen H S, Feuer E J, Cronin K A (eds). SEER Cancer Statistics Review, 1975-2015, National Cancer Institute. Bethesda, Md., https://seer.cancer.gov/csr/1975 2015/, based on November 2017 SEER data submission, posted to the SEER web site, April 2018.
2. Deorah S, Lynch C F, Sibenaller Z A, Ryken T C. Trends in brain cancer incidence and survival in the United States: surveillance, epidemiology, and end results program, 1973 to 2001. Neurosurg. Focus, 2006; 20:E1.
3. Chi A S, Norden A D, Wen P Y. Antiangiogenic strategies for treatment of malignant gliomas. Neurotherapeutics. 2009; 6:513-526.
4. Hervey-Jumper S L, Berger M S. Role of surgical resection in low- and high-grade gliomas. Curr Treat Options Neurol. 2014; 16:284.
5. Ius T, Isola M, Budai R, Pauletto G, Tomasino B, Fadiga L, Skrap M. Low-grade glioma surgery in eloquent areas: volumetric analysis of extent of resection and its impact on overall survival. A single-institution experience in 190 patients: clinical article. J Neurosurg. 2012; 117:1039-1052.
6. Eyipoglu I Y, Buchfelder M, Savaskan N E. Surgical resection of malignant gliomas-role in optimizing patient outcome. Nat Rev Neurol. 2013; 9:141-151.
7. Bloch O, Han S J, Cha S, Sun M Z, Aghi M K, McDermott M W, Berger M S, Parsa A T. Impact of extent of resection for recurrent glioblastoma on overall survival: clinical article. J Neurosurg. 2012; 117:1032-1038.
8. Beiko J, Suki D, Hess K R, Fox B D, Cheung V, Cabral M, Shonka N, Gilbert M R, Sawaya R, Prabhu S S, Weinberg J, Lang F F, Aldape K D, Sulman E P, Rao G, McCutcheon I E, Cahill D P. IDH1 mutant malignant astrocytomas are more amenable to surgical resection and have a survival benefit associated with maximal surgical resection. Neuro Oncol. 2014; 16:81-91.
9. Kubben P L, ter Meulen K J, Schijns O E, ter Laak-Poort M P, van Overbeeke J J, van Santbrink H. Intraoperative MRI-guided resection of glioblastoma multiforme: a systematic review. Lancet Oncol. 2011; 12:1062-1070.
10. Hefti M, von Campe G, Moschopulos M, Siegner A, Looser H, Landolt H. 5-aminolaevulinic acid-induced protoporphyrin IX fluorescence in high-grade glioma surgery: A one-year experience at a single institution. Swiss Med Wkly. 2008; 138:180-185.
11. Pichlmeier U, Bink A, Schackert G, Stummer W, Group tAGS. Resection and survival in glioblastoma multiforme: An RTOG recursive partitioning analysis of ALA study patients. Neuro-Oncology. 2008; 10:1025-1034.
12. Stummer W, Pichlmeier U, Meinel T, Wiestler O D, Zanella F, Reulen H-J, Group ftA-G S. Fluorescence-guided surgery with 5-aminolevulinic acid for resection of malignant glioma: a randomised controlled multicentre phase III trial. Lancet Oncology. 2006; 7:392-401.
13. Chung I W, Eljamel S. Risk factors for developing oral 5-aminolevulinic acid-induced side effects in patients undergoing fluorescence guided resection. Photodiagnosis Photodyn Ther. 2013; 10:362-367.
14. Eyipoglu I Y, Hore N, Savaskan N E, Grummich P, Roessler K, Buchfelder M, Ganslandt O. Improving the extent of malignant glioma resection by dual intraoperative visualization approach. PLoS One. 2012; 7:e44885.
15. Stummer W, Pichlmeier U, Meinel T, Wiestler O D, Zanella F, Reulen H J, Group A-G S. Fluorescence-guided surgery with 5-aminolevulinic acid for resection of malignant glioma: a randomised controlled multicentre phase III trial. Lancet Oncol. 2006; 7:392-401.
16. Thurber G M, Figueiredo J-L, Weissleder R. Detection limits of intraoperative near infrared imaging for tumor resection. Journal of Surgical Oncology. 2010; 102:758-764.
17. Stroud M R, Hansen S J, Olson J M. In vivo bio-imaging using chlorotoxin-based conjugates. Curr Pharm Des. 2011; 17:4362-4371.
18. Wu X-S, Jian X-C, Yin B, He Z-J. Development of the research on the application of chlorotoxin in imaging diagnostics and targeted therapies for tumors. Chinese Journal of Cancer. 2010; 29:626-630.
19. Mamelak A N, Jacoby D B. Targeted delivery of antitumoral therapy to glioma and other malignancies with synthetic chlorotoxin (TM-601). Expert Opin Drug Deliv. 2007; 4:175-186.
20. Kesavan K, Ratliff J, Johnson E W, Dahlberg W, Asara J M, Misra P, Frangioni J V, Jacoby D B. Annexin A2 is a molecular target for TM601, a peptide with tumor-targeting and anti-angiogenic effects. J Biol Chem. 2010; 285:4366-4374.
21. Dardevet L, Rani D, Abd El Aziz T, Bazin I, Sabatier J-M, Fadl M, Brambilla E, De Waard M. Chlorotoxin: A helpful natural scorpion peptide to diagnose glioma and fight tumor invasion. Toxins 2015:7:1079-1101.
22. Hockaday D C, Shen S, Fiveash J, Raubitschek A, Colcher D, Liu A, Alvarez V, Mamelak A N. Imaging glioma extent with ¹³¹I-TM-601. The Journal of Nuclear Medicine. 2005; 46:580-586.
23. Mamelak A N, Rosenfeld S, Bucholz R, Raubitschek A, Nabors L B, Fiveash J B, Shen S, Khazaeli M B, Colcher D, Liu A, Osman M, Guthrie B, Schade-Bijur S, Hablitz D M, Alvarez V L, Gonda M A. Phase I single-dose study of intracavitary-administered iodine-131-TM-601 in adults with recurrent high-grade glioma. Journal of Clinical Oncology. 2006; 24:3644-3650.
24. Gribbin T, Senzer N, Raizer J, Shen S, Nabors L, Wiranowska M, Fiveash J. A phase I evaluation of intravenous (I V) ̂131 I-chlorotoxin delivery to solid peripheral and intracranial tumors. J Clin Oncol. 2009; 27 (suppl):abstr e14507.
25. Butte P V, Mamelak A, Parrish-Novak J, Drazin D, Shweikeh F, Gangalum P R, Chesnokova A, Ljubimova J Y, Black K. Near-infrared imaging of brain tumors using the Tumor Paint BLZ-100 to achieve near-complete resection of brain tumors. Neurosurg Focus. 2014; 36:E1.
26. Veiseh M, Gabikian P, Bahrami S-B, Veiseh O, Zhang M, Hackman R C, Ravanpay A C, Stroud M R, Kusuma Y, Hansen S J, Kwok D, Munoz N M, Sze R W, Grady W M, Greenberg N M, Ellenbogen R G, Olson J M. Tumor Paint: A chlorotoxin:Cy5.5 bioconjugate for intraoperative visualization of cancer foci. Cancer Res. 2007; 67:6882-6888.
27. Huang R, Vider J, Kovar J L, Olive D M, Mellinghoff I K, Mayer-Kuckuk P, Kircher M F, Blasberg R G. Integrin av83-targeted IRDye 800C W near-infrared imaging of glioblastoma. Clin Cancer Res. 2012; 18:5731-5740.
28. Lyons S A, O'Neal J, Sontheimer H. Chlorotoxin, a scorpion-derived peptide, specifically binds to gliomas and tumors of neuroectodermal origin. Glia. 2002; 39:162-73.
29. Soroceanu L, Gillespie Y, Khazaeli M B, Sontheimer H. Use of chlorotoxin for targeting of primary brain tumors. Cancer Res. 1998; 58:4871-4879.
30. Ljubimova J Y, Portilla-Arias J, Patil R, Ding H, Inoue S, Markman J L, Rekechenetskiy A, Konda B, Gangalum P R, Chesnokova A, Ljubimov A V, Black K L, Holler E. Toxicity and efficacy evaluation of multiple targeted polymalic acid conjugates for triple-negative breast cancer treatment. J Drug Target. 2013; 21:956-967.
31. Ljubimova J Y, Ding H, Portilla-Arias J, Patil R, Gangalum P R, Chesnokova A, Inoue S, Rekechenetskiy A, Nassoura T, Black K L, Holler E. (2014) Polymalic acid-based nano biopolymers for targeting of multiple tumor markers: an opportunity for personalized medicine? J Vis Exp; (88).
32. Lee B S, Fujita M, Khazenzon N M, Wawrowsky K A, Wachsmann-Hogiu S, Farkas D L, Black K L, Ljubimova J Y, Holler E. Polycefin, a new prototype of a multifunctional nanoconjugate based on poly(beta-L-malic acid) for drug delivery. Bioconjug. Chem. 2006; 17:317-326.
33. Ding H, Inoue S, Ljubimov A V, Patil R, Portilla-Arias J, Hu J, Konda B, Wawrowsky K A, Fujita M, Karabalin N, Sasaki T, Black K L, Holler E, Ljubimova J Y. Inhibition of brain tumor growth by intravenous poly (B-L-malic acid) nanobioconjugate with pH-dependent drug release [corrected]. Proc Natl Acad Sci USA. 2010; 107:18143-18148.
34. Inoue S, Patil R, Portilla-Arias J, Ding H, Konda B, Espinoza A, Mongayt D, Markman J L, Elramsisy A, Phillips H W, Black K L, Holler E, Ljubimova J Y. Nanobiopolymer for direct targeting and inhibition of EGFR expression in triple negative breast cancer. PLoS One. 2012; 7:e31070.
35. Patil R, Ljubimov A V, Gangalum P R, Ding H, Portilla-Arias J, Wagner S, Inoue S, Konda B, Rekechenetskiy A, Chesnokova A, Markman J L, Ljubimov V A, Li D, Prasad R S, Black K L, Holler E, Ljubimova J Y. MRI virtual biopsy and treatment of brain metastatic tumors with targeted nanobioconjugates: nanoclinic in the brain. ACS Nano, 2015; 9:5594-5608.
36. Ding H, Gangalum P R, Galstyan A, Fox I, Patil R, Hubbard P, Murali R, Ljubimova J Y and Holler E. HER2-positive breast cancer targeting and treatment by a peptide-conjugated mini nanodrugs” Nanomedicine: Nanotechnology, Biology, and Medicine 2017; 13:631-639.
37. Patil R, Gangalum P R, Wagner S, Portilla-Arias J, Ding H, Rekechenetskiy A, Konda B, Inoue S, Black K L, Ljubimova J Y, Holler E. (2015) Curcumin Targeted, Polymalic Acid-Based MRI Contrast Agent for the Detection of A B Plaques in Alzheimer's Disease. Macromol Biosci. 15:1212-1217.
38. Patil R, Portilla-Arias J, Ding H, Inoue S, Konda B, Hu J, Wawrowsky K A, Shin P K, Black K L, Holler E, and Ljubimova J Y. Temozolomide delivery to tumor cells by a multifunctional nano vehicle based on poly(B-L-malic acid). Pharm Res. 2010; 27: 2317-29.
39. Patil, R, Portilla-Arias J, Ding H, Konda B, Black K L, Holler E, and Ljubimova J Y. Cellular Delivery of Doxorubicin via pH-Controlled Hydrazone Linkage Using Multifunctional Nano Vehicle Based on Poly(B-L-malic Acid). Int. J. Mol. Sci 2012; 13:11681-93 2012 (Special issue: Bioactive Nanoparticles)
40. Bertrand N, Wu J, Xu X, Kamaly N, Farokhzad O C. Cancer nanotechnology: the impact of passive and active targeting in the era of modern cancer biology. Adv Drug Deliv Rev. 2014; 66:2-25.
41. Ding H, Patil R, Portilla-Arias J, Black K L, Ljubimova J Y, Holler E. Quantitative analysis of PMLA nanoconjugate components after backbone cleavage. Int J Mol Sci. 2015; 16:8607-8620.
42. Zhou Y, Kim Y S, Milenic D E, Baidoo K E, Brechbiel M W. In vitro and in vivo analysis of indocyanine green-labeled panitumumab for optical imaging—a cautionary tale. Bioconjug Chem 2010; 25: 1801-1810.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. An imaging nanoagent comprising a polymalic acid-based molecular scaffold, a chlorotoxin peptide or variant thereof, and at least one fluorescent moiety, wherein the chlorotoxin peptide and the at least one fluorescent moiety are covalently linked to the polymalic acid-based molecular scaffold.

2. The imaging nanoagent of claim 1, wherein the at least one fluorescent moiety is a cyanine moiety.

3. The imaging nanoagent of claim 2, wherein the at least one fluorescent moiety comprises an indocyanine green (ICG) or Rhodamine.

4. The imaging nanoagent of claim 1, wherein the chlorotoxin peptide or variant thereof comprises an amino acid sequence with at least 90% sequence identity to the sequence selected from the group consisting of: SEQ ID NOs: 1-10, and binds to cancerous cells.

5. The imaging nanoagent of claim 1, wherein the chlorotoxin peptide or variant thereof is linked to the polymalic acid based molecular scaffold by a linker.

6. The imaging nanoagent of claim 5, wherein the linker comprises a polyethylene glycol (PEG).

7. The imaging nanoagent of claim 1 further comprising at least one biologically active molecular module.

8. The imaging nanoagent of claim 7, wherein the at least one fluorescent moiety further comprises at least two fluorescent moieties interspaced with the at least one biologically active molecular module.

9. The imaging nanoagent of claim 7, wherein the at least one biologically active molecular module is selected from the group consisting of: an anti-cancer agent, a targeting ligand, and an endosomolytic ligand.

10. The imaging nanoagent of claim 9, wherein the at least one biologically active molecular module is the endosomolytic ligand covalently linked with the polymalic acid-based molecular scaffold.

11. The imaging nanoagent of claim 10, wherein the endosomolytic ligand comprises a plurality of leucine or valine residues.

12. The imaging nanoagent of claim 10, wherein the endosomolytic ligand is Leu-Leu-Leu (LLL).

13. The imaging nanoagent of claim 7, wherein the at least one biologically active molecular module is an anti-cancer agent selected from the group consisting of: an antisense oligonucleotide, an siRNA oligonucleotide, an antibody, a polypeptide, an oligopeptide, a low molecular weight drug, radioisotope, toxin, cytotoxic agent, enzyme, sensitizing drug, nucleic acid, anti-angiogenic agent, cisplatin, anti-metabolite, mitotic inhibitor, growth factor inhibitor, paclitaxel, temozolomide, topotecan, fluorouracil, vincristine, vinblastine, procarbazine, dacarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane, amifostine or a combination thereof.

14. The imaging nanoagent of claim 7, wherein the at least one biologically active molecular module comprises at least two different anti-cancer agents covalently linked to the polymalic acid-based molecular scaffold.

15. A pharmaceutically acceptable composition comprising the imaging nanoagent of claim 1 and a pharmaceutically acceptable carrier or excipient.

16. A method for detecting and removing a cancer comprising:
administering an imaging nanoagent comprising a polymalic acid-based molecular scaffold, a chlorotoxin peptide or variant thereof, and at least one fluorescent moiety, wherein the chlorotoxin peptide and the at least one fluorescent moiety are covalently linked to the polymalic acid-based molecular scaffold;
detecting the presence or absence of the imaging nanoagent, wherein the presence of the imaging nanoagent in the cells or tissues indicate the presence of cancerous cells or tissue; and
surgically removing the cancerous cell or tissue.

17. The method of claim 16, wherein the imaging nanoagent is included in a pharmaceutically acceptable composition comprising a pharmaceutically acceptable carrier or excipient.

18. The method of claim 16, wherein the at least one fluorescent moiety is a cyanine moiety.

19. The method of claim 18, wherein the at least one fluorescent moiety comprises an indocyanine green (ICG) or Rhodamine.

20. The method of claim 16, wherein the chlorotoxin peptide or variant thereof comprises an amino acid sequence with at least 90% sequence identity to the sequence selected from the group consisting of: SEQ ID NOs: 1-10, and binds to cancerous cells.

21. The method of claim 16, wherein the chlorotoxin peptide or variant thereof is linked to the polymalic acid-based molecular scaffold by a linker.

22. The method of claim 16, further comprising at least one biologically active molecular module.

23. The method of claim 22, wherein the imaging nanoagent comprises at least two fluorescent moieties interspaced with the at least one biologically active module.

24. The method of claim 22, wherein the at least one biologically active molecular module is selected from the group consisting of: an anti-cancer agent, a targeting ligand, and an endosomolytic ligand.

25. The method of claim 24, wherein the at least one biologically active molecular module is the endosomolytic ligand covalently linked with the polymalic acid-based molecular scaffold.

26. The method of claim 25, wherein the endosomolytic ligand comprises a plurality of leucine or valine residues.

27. The method of claim 16, wherein the step of detecting comprises visualizing the imaging nanoagent.

28. The method of claim 27, wherein the visualizing is performed in vivo.

29. The method of claim 28, wherein the visualizing includes imaging a tissue in a brain of the subject.

30. The method of claim 16, wherein the cancer is primary cancer, a metastatic cancer or both.

31. The method of claim 30, wherein the primary cancer is selected from the group consisting of: brain, lung, head and neck cancers, and melanoma.

32. The method of claim 16, wherein the subject is a mammal.

33. The method of claim 32, wherein the mammal is selected from the group consisting of: a rodent, an experimental human-breast tumor-bearing nude mouse and a human.

34. A method for treating cancer in a subject, comprising performing the method of claim 16.

35. The method of claim 34, wherein the method further comprises administering an additional anti-cancer therapy to the subject.

36. The method of claim 35, wherein the additional anti-cancer therapy is selected from the group consisting of: chemotherapy, radiation therapy, thermotherapy, immunotherapy, hormone therapy, laser therapy, anti-angiogenic therapy, and any combinations thereof.

37. An imaging nanoagent comprising:
a polymalic acid-based molecular scaffold;
a chlorotoxin peptide covalently linked to the polymalic acid-based molecular scaffold by a polyethylene glycol (PEG) linker;
a plurality of cyanine moieties covalently linked to the polymalic acid-based molecular scaffold; and
at least one biological active molecular module covalently linked to the polymalic acid-based molecular scaffold, wherein the at least one biological active molecular module is selected from the group consisting of an anti-cancer agent, a targeting ligand, and an endosomolytic ligand, and the plurality of the cyanine moieties are interspaced with the chlorotoxin peptide, the at least one biologically active molecular module, or a combination thereof.
